(19) United States
(12) Patent Application Publication
(10) Pub. No.: US 2024/0175893 A1
(43) Pub. Date: May 30, 2024

(54) INFORMATION PROCESSING APPARATUS, INFORMATION PROCESSING METHOD, AND PROGRAM

(71) Applicant: SONY GROUP CORPORATION, TOKYO (JP)

(72) Inventors: MASATO KIMISHIMA, TOKYO (JP); KOSEI YAMASHITA, TOKYO (JP); TETSURO SATO, TOKYO (JP)

(21) Appl. No.: 18/569,284

(22) PCT Filed: Feb. 9, 2022
(86) PCT No.: PCT/JP2022/005155

§ 371 Date: Dec. 12, 2023

(30) Foreign Application Priority Data
Jun. 22, 2021 (JP) 2021-102970

(51) Int. Cl. G01P 7/00; G01C 19/00; G01P 15/18; G06N 5/04; G06N 20/00

(52) U.S. Cl. CPC G01P 7/00; G01C 19/00; G01P 15/18; G06N 5/04; G06N 20/00

(57) ABSTRACT

The present disclosure relates to an information processing apparatus, an information processing method, and a program capable of implementing highly accurate motion capture.

The posture, speed, and position of the sensor unit are calculated by integrating the angular velocity and the acceleration detected by the sensor unit. Since at least one of the calculated posture, speed, or position of the sensor unit is obtained by integration, the accuracy decreases with the lapse of time, and thus an inference parameter for inferring at least one of the posture, speed, or position of the sensor unit is learned using only a value having a small error with an elapsed time shorter than a predetermined time. The present invention can be applied to a motion capture apparatus.

### TECHNICAL FIELD

The present disclosure relates to an information processing apparatus, an information processing method, and a program, and more particularly, to an information processing apparatus, an information processing method, and a program capable of implementing highly accurate motion capture.

### BACKGROUND ART

Although motion capture techniques for detecting a motion of a person have been proposed, a highly accurate position detection technique is required to implement a highly accurate motion capture technology.

As a highly accurate position detection technique, for example, a technique has been proposed in which, in measurement of a walking movement amount, a measurement result with an accuracy lower than a predetermined accuracy with an elapsed time from the start of measurement is not used, and a movement amount is measured using a measurement result with an accuracy lower than a predetermined accuracy (see Patent Document 1).

### CITATION LIST

### Patent Document

Patent Document 1: Japanese Patent Application Laid-Open No. 2013-210866

### SUMMARY OF THE INVENTION

### Problems to be Solved by the Invention

However, in the technique described in Patent Document 1, only the measurement result until the accuracy decreases with the elapsed time from the start of measurement is used, and when a predetermined time or more has elapsed, highly accurate measurement cannot be performed.

Furthermore, it is conceivable to perform learning for implementing position detection in advance and perform detection with high accuracy. However, in order to enable detection with high accuracy even when used by all people in motion capture or the like, an enormous amount of learning data is required, which increases labor and cost.

The present disclosure has been made in view of such a situation, and in particular, an object of the present disclosure is to achieve high accuracy of position detection by a simple method and to implement highly accurate motion capture.

### Solutions to Problems

An information processing apparatus and a program according to one aspect of the present disclosure are an information processing apparatus and a program including: a calculation unit that calculates information to be a learning label on the basis of a sensor value including an angular velocity and an acceleration detected by a sensor unit; a learning unit that learns an inference parameter for inferring at least one of a posture, a speed, or a position of the sensor unit on the basis of the information to be the learning label calculated by the calculation unit and the sensor value; and a learning label supply unit that supplies, to the learning unit, information to be the learning label with higher accuracy than predetermined accuracy among the information to be the learning label calculated by the calculation unit, in which the learning unit learns the inference parameter for inferring at least one of the posture, the speed, or the position of the sensor unit on the basis of the information to be the learning label with higher accuracy than the predetermined accuracy supplied from the learning label supply unit and the sensor value.

An information processing method according to one aspect of the present disclosure is an information processing method of an information processing apparatus including: a calculation unit; a learning unit; and a learning label supply unit, in which the calculation unit calculates information to be a learning label on the basis of a sensor value including an angular velocity and an acceleration detected by a sensor unit, the learning unit learns an inference parameter for inferring at least one of a posture, a speed, or a position of the sensor unit on the basis of the information to be the learning label calculated by the calculation unit and the sensor value, the learning label supply unit supplies, to the learning unit, information to be the learning label with higher accuracy than predetermined accuracy among the information to be the learning label calculated by the calculation unit, and the learning unit learns the inference parameter for inferring at least one of the posture, the speed, or the position of the sensor unit on the basis of the information to be the learning label with higher accuracy than the predetermined accuracy supplied from the learning label supply unit and the sensor value.

In one aspect of the present disclosure, information to be a learning label is calculated on the basis of a sensor value including an angular velocity and an acceleration detected by a sensor unit, an inference parameter for inferring at least one of a posture, a speed, or a position of the sensor unit is learned on the basis of the calculated information to be the learning label and the sensor value, information to be the learning label with higher accuracy than predetermined accuracy is supplied to be used for learning among the calculated information to be the learning label, and the inference parameter for inferring at least one of the posture, the speed, or the position of the sensor unit is learned on the basis of the information to be the learning label with higher accuracy than the predetermined accuracy and the sensor value.

### BRIEF DESCRIPTION OF DRAWINGS

FIG. 1 is a diagram for explaining a configuration example of an inertial navigation apparatus.

FIG. 2 is a diagram for explaining accumulation of errors.

FIG. 3 is a diagram for explaining an appearance configuration of a motion capture system of the present disclosure.

FIG. 4 is a block diagram illustrating a configuration of a motion capture system of the present disclosure.

FIG. 5 is a diagram for explaining a configuration example of a first embodiment of the electronic device and the sensor unit in FIGS. 3 and 4.

FIG. 6 is a flowchart for explaining learning processing by the electronic device in FIG. 5.

FIG. 7 is a flowchart for explaining a first modification of the learning processing by the electronic device in FIG. 5.

FIG. 8 is a flowchart for explaining a second modification of the learning processing by the electronic device in FIG. 5.

FIG. 9 is a flowchart for explaining inference processing by the electronic device in FIG. 5.

FIG. 10 is a flowchart for explaining a modification of inference processing by the electronic device in FIG. 5.

FIG. 11 is a diagram for explaining a configuration example of a second embodiment of the electronic device in FIGS. 3 and 4.

FIG. 12 is a flowchart for explaining learning processing by the electronic device in FIG. 11.

FIG. 13 is a diagram for explaining an example of continuing learning of an inference parameter without stopping.

FIG. 14 is a diagram for explaining an example of continuing learning of an inference parameter without stopping.

FIG. 15 is a diagram for explaining a configuration example of a third embodiment of the electronic device in FIGS. 3 and 4.

FIG. 16 is a flowchart for explaining learning processing by the electronic device in FIG. 15.

FIG. 17 is a diagram for explaining a configuration example of a fourth embodiment of the electronic device in FIGS. 3 and 4.

FIG. 18 is a flowchart for explaining learning processing by the electronic device in FIG. 17.

FIG. 19 illustrates a configuration example of a general-purpose computer.

### MODE FOR CARRYING OUT THE INVENTION

Preferred embodiments of the present disclosure are hereinafter described in detail with reference to the accompanying drawings. Note that, in the present specification and the drawings, components having substantially the same functional configurations are denoted using the same reference signs, and redundant descriptions are omitted.

Hereinafter, embodiments for carrying out the present technology will be described. The description will be given in the following order.

1. Principle of Inertial Navigation

2. First Embodiment

3. Second Embodiment

4. Third Embodiment

5. Fourth Embodiment

6. Example of Execution by Software

### 1. Principle of Inertial Navigation

In particular, the present disclosure is intended to achieve high accuracy of a position detection technique and implement highly accurate motion capture.

In describing the technology for implementing highly accurate motion capture of the present disclosure, first, the principle of inertial navigation will be described.

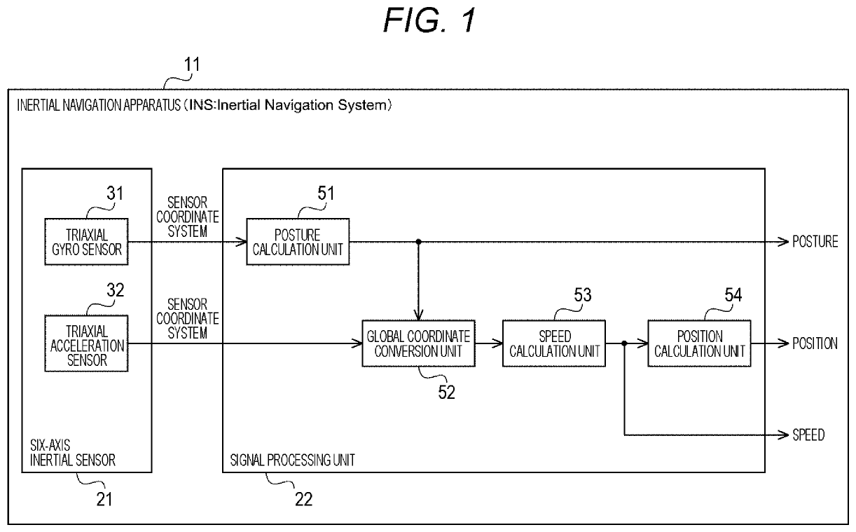

FIG. 1 illustrates a configuration example of a general inertial navigation apparatus. An inertial navigation apparatus 11 in FIG. 1 is mounted on various moving bodies such as an aircraft and a drone, and detects the posture, speed, and position of the moving body in a three-dimensional space.

Note that the three-dimensional space here is, for example, a space in which a position in the space can be expressed by coordinates on each of the x axis, the y axis, and the z axis. Hereinafter, the three-dimensional space is also referred to as an xyz space including three axes of an x axis, a y axis, and a z axis.

The inertial navigation apparatus 11 includes a six-axis inertial sensor 21 and a signal processing unit 22. The six-axis inertial sensor 21 detects the triaxial angular velocities and the triaxial accelerations in the three-dimensional space, and outputs them to the signal processing unit 22.

The signal processing unit 22 detects a posture, a speed, and a position in a three-dimensional space of the moving body on which the inertial navigation apparatus 11 is mounted on the basis of the triaxial angular velocities and the triaxial accelerations detected by the six-axis inertial sensor 21.

More specifically, the six-axis inertial sensor 21 includes a triaxial gyro sensor 31 and a triaxial acceleration sensor 32.

The triaxial gyro sensor 31 detects angular velocity of each of the xyz axes in the sensor coordinate system and outputs the detected angular velocity to the signal processing unit 22.

The triaxial acceleration sensor 32 detects acceleration of each of the xyz axes in the sensor coordinate system and outputs the acceleration to the signal processing unit 22.

The signal processing unit 22 includes a posture calculation unit 51, a global coordinate conversion unit 52, a speed calculation unit 53, and a position calculation unit 54.

When acquiring the information of the angular velocity in the sensor coordinate system supplied from the triaxial gyro sensor 31 of the six-axis inertial sensor 21, the posture calculation unit 51 performs integration, obtains an angle with respect to three axes indicating the posture in the global coordinate system, and outputs the obtained angle.

The global coordinate conversion unit 52 converts the information of the accelerations in the xyz axes in the sensor coordinate system supplied from the triaxial acceleration sensor 32 of the six-axis inertial sensor 21 into the information of the acceleration in the global coordinate system on the basis of the information of the angle indicating the posture in the global coordinate system obtained in the posture calculation unit 51, and outputs the information to the speed calculation unit 53.

The speed calculation unit 53 calculates and outputs the speed in the global coordinate system by integrating the acceleration in the global coordinate system.

The position calculation unit 54 calculates and outputs the position in the global coordinate system by integrating the speed in the global coordinate system calculated by the speed calculation unit 53.

As a result, the posture (angle), speed, and position in the global coordinate system of the moving body on which the inertial navigation apparatus 11 is mounted are detected.

As a result, it is possible to detect the posture, speed, and position in the global coordinate system with predetermined accuracy in various applications without depending on the motion model.

### Accumulation of Errors

However, in a case where the inertial navigation apparatus 11 described with reference to FIG. 1 is used, the triaxial angular velocities and the triaxial accelerations detected by the six-axis inertial sensor 21 are integrated, and the posture, the speed, and the position continue to be obtained. Therefore, errors are accumulated as time passes.

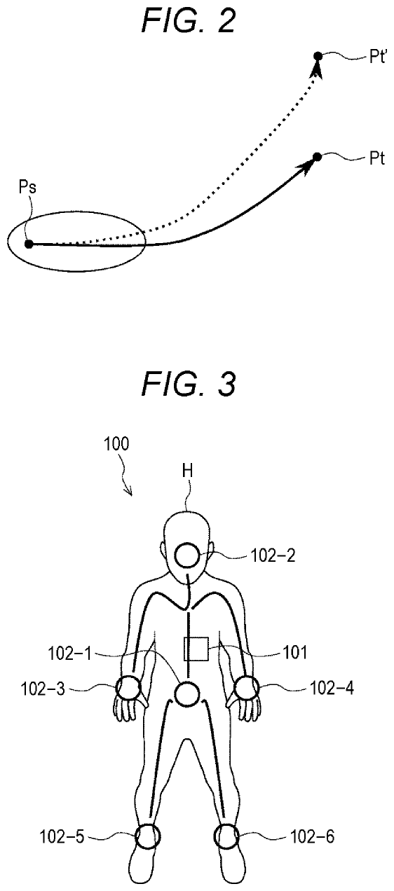

As a result, in reality, even in a case where the moving body on which the inertial navigation apparatus 11 is mounted moves from the start point Ps to the point Pt along the path of the arrow indicated by the solid line in FIG. 2, there is a possibility that the detection result is detected as if the moving body has moved to, for example, the point Pt′ indicated by the dotted arrow in FIG. 2 due to accumulation of the error, and the error gradually increases as time passes.

Therefore, in the present disclosure, attention is paid to a range in the vicinity of the start point Ps, that is, a range in which an error between the path indicated by the solid arrow and the path indicated by the dotted arrow in the range surrounded by the solid line in FIG. 2 is small. That is, at least one of the posture, the speed, or the position obtained by the inertial navigation within the range surrounded by the solid line in FIG. 2 is set as a correct answer label, and the inference parameter is obtained by machine learning using the detection result of the six-axis inertial sensor 21 as an input.

Then, the posture, the speed, and the position based on the detection result of the six-axis inertial sensor 21 are inferred using the inference parameters obtained by learning, thereby implementing highly accurate positioning.

By applying such a high-precision positioning technology to motion capture, highly accurate motion capture is implemented.

Furthermore, the inference parameter is generated by learning based on a motion when the six-axis inertial sensor 21 is actually attached to an individual who uses motion capture, so that the inference parameter can be personalized.

As a result, a large amount of learning data required for learning of the inference parameter that increases the accuracy even if used by all people is unnecessary, the learning of the inference parameter can be simplified, and highly accurate motion capture can be implemented by more simple processing.

Furthermore, since an individual who uses motion capture only wears and uses the above-described six-axis inertial sensor 21, for example, it is possible to learn a highly accurate inference parameter with a simpler configuration than an apparatus configuration in which an image captured by a camera is used as a correct answer label.

### 2. First Embodiment

### Configuration Example of Motion Capture System

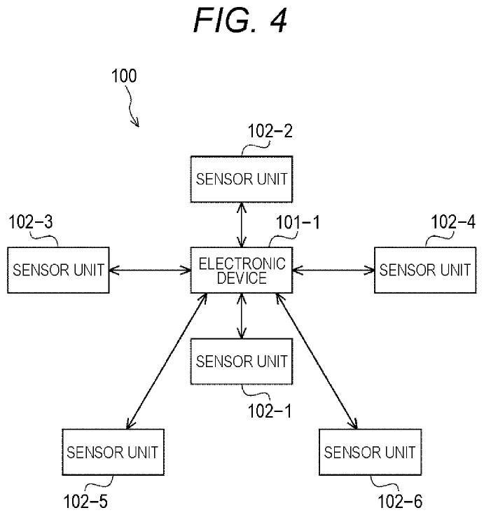

Next, a configuration example of a motion capture system to which the technology of the present disclosure is applied will be described with reference to FIGS. 3 and 4.

FIG. 3 illustrates an appearance of the motion capture system 100 of the present disclosure, and FIG. 4 is a block diagram for explaining functions implemented by a motion capture system 100.

The motion capture system 100 detects the motion (operation) of the user H on the basis of the posture, speed, and position of each motion of the torso, head, and limbs of the human body that is the user H.

The motion capture system 100 includes an electronic device 101 and sensor units 102-1 to 102-6.

The electronic device 101 is, for example, a portable information processing apparatus such as a smartphone, and is configured to be able to communicate with each of the sensor units 102-1 to 102-6 in a wired manner or a wireless manner such as Bluetooth (registered trademark) or WiFi.

The electronic device 101 acquires sensor values that are detection results of the sensor units 102-1 to 102-6, calculates the respective postures, speeds, and positions, and learns inference parameters for inferring the respective postures, speeds, and positions on the basis of the respective sensor values of the sensor units 102-1 to 102-6 by machine learning based on the respective sensor values of the sensor units 102-1 to 102-6 and at least one of the respective calculated postures, speeds, or positions.

In addition, the electronic device 101 outputs the inference results of the posture, the speed, and the position on the basis of the sensor values of the sensor units 102-1 to 102-6 using the learned inference parameters.

The sensor units 102-1 to 102-6 have a configuration corresponding to the six-axis inertial sensor 21 in FIG. 1, and are fixed to the torso, the head, the right and left wrists, and the right and left ankles of the human body as the user H with a band or the like, detect the triaxial angular velocities and the triaxial accelerations at the respective positions, and supply the detected angular velocities and accelerations to the electronic device 101.

Note that, in this example, the electronic device 101 infers the posture, the speed, and the position of each of the sensor units 102-1 to 102-6 in the global coordinate system from the learned inference parameter on the basis of the sensor values of the sensor units 102-1 to 102-6.

Then, on the basis of the inferred posture, speed, and position of the sensor units 102-1 to 102-6, the electronic device 101 detects, as motion of the user H, the relative speed and position of each of the sensor unit 102-2 fixed to the head, the sensor units 102-3 and 102-4 fixed to the right and left wrists, and the sensor units 102-5 and 102-6 fixed to the right and left ankles, with respect to the position of the sensor unit 102-1 fixed to the body portion of the user H.

In this example, the sensor unit 102-1 fixed to the body portion and the electronic device 101 are configured separately, but may be integrated as a smartphone, for example. The sensor units 102-2 to 102-6 may also be configured by, for example, a smart headset, a smart watch (for the ankle, the smart anklet), or the like each integrated with a configuration corresponding to the electronic device 101.

Furthermore, in a case where the sensor units 102-1 to 102-6 do not need to be particularly distinguished, they are also simply referred to as a sensor unit 102, and it similarly applies to other configurations.

### Detailed Configuration Example of Electronic Device and Sensor Unit

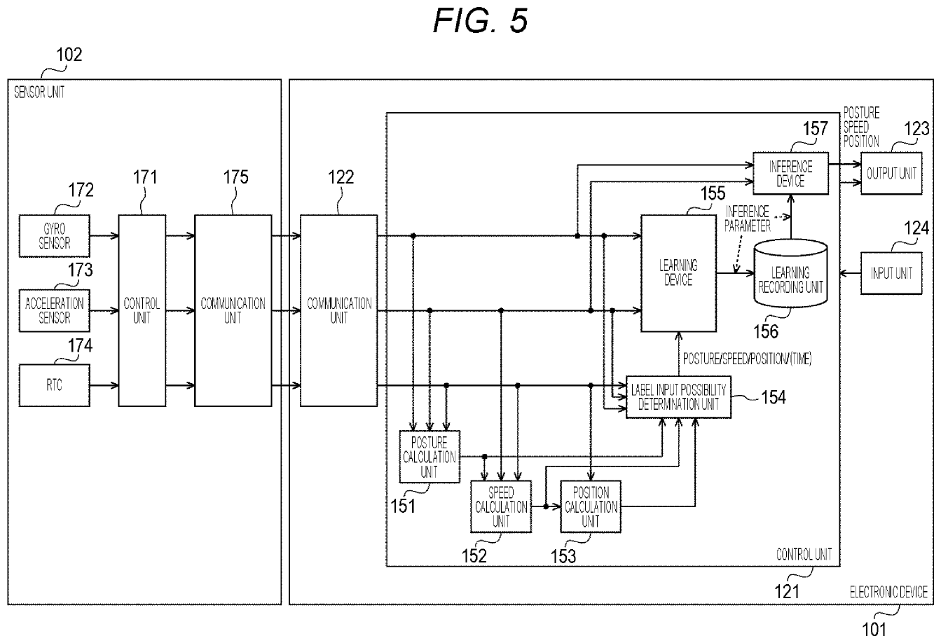

Next, configuration examples of the electronic device 101 and the sensor unit 102 will be described with reference to FIG. 5.

The sensor unit 102 includes a control unit 171, a gyro sensor 172, an acceleration sensor 173, a real time clock (RTC) 174, and a communication unit 175.

The control unit 171 includes a processor and a memory, operates on the basis of various data and programs, and controls the entire operation of the sensor unit 102.

The gyro sensor 172 has a configuration corresponding to the triaxial gyro sensor 31 in FIG. 1, detects the triaxial angular velocities in the sensor coordinate system of the sensor unit 102, and outputs the angular velocities to the control unit 171.

The acceleration sensor 173 has a configuration corresponding to the triaxial acceleration sensor 32 of FIG. 1, detects the triaxial accelerations in the sensor coordinate system of the sensor unit 102, and outputs the accelerations to the control unit 171.

The real time clock (RTC) 174 generates time information (time stamp) and outputs the time information to the control unit 171.

The control unit 171 associates the triaxial angular velocities in the sensor coordinate system supplied from the gyro sensor 172, the triaxial accelerations in the sensor coordinate system supplied from the acceleration sensor 173, and the time information supplied from the RTC 174 with each other, controls the communication unit 175, and transmits the time information to the electronic device 101.

The communication units 175 and 122 exchanges data and programs with each other by wired communication or wireless communication by Bluetooth (registered trademark), wireless LAN, or the like.

Note that, in FIG. 5, three arrows connecting between the communication units 175 and 122 in the electronic device 101 and the sensor unit 102 and the front and rear thereof in the drawing represent paths to which angular velocities in three axial directions in the sensor coordinate system, accelerations in three axial directions in the sensor coordinate system, and time information (time stamps) are respectively supplied from the top. Note that the communication units 175 and 122 can communicate with each other, and can also communicate from the communication unit 122 to the communication unit 175 although the direction of the arrow is not illustrated.

The electronic device 101 includes a control unit 121, a communication unit 122, an output unit 123, and an input unit 124.

The control unit 121 includes a processor and a memory, operates on the basis of various data and programs, and executes various data and programs to control the entire operation of the electronic device 101.

More specifically, the control unit 121 controls the communication unit 122 to acquire the triaxial angular velocities and the triaxial accelerations in the sensor coordinate system supplied from the sensor unit 102, and the time information (time stamp) of the corresponding detection result.

The control unit 121 calculates the posture, speed, and position of the sensor unit 102 on the basis of the acquired the triaxial angular velocities and the triaxial accelerations in the sensor coordinate system supplied from the sensor unit 102, and time information (time stamp) of a corresponding detection result.

Furthermore, the control unit 121 uses at least one of the posture, the speed, or the position of the sensor unit 102, which is a calculation result, as a learning label, which is a correct answer label, and uses the triaxial angular velocities and the triaxial accelerations supplied from the sensor unit 102 as inputs to learn inference parameters for inferring the posture, the speed, and the position of the sensor unit 102 by machine learning such as a neural network.

Then, the control unit 121 infers the posture, speed, and position of the sensor unit 102 from the inference parameter serving as the learning result and the input of the triaxial angular velocities and the triaxial accelerations supplied from the sensor unit 102, controls the output unit 123 including a display and a speaker to output the inference result, and presents the result to the user.

Furthermore, in learning, the control unit 121 stops learning of the inference parameter when the triaxial angular velocities and the triaxial accelerations supplied from the sensor unit 102 that do not satisfy the predetermined accuracy are supplied.

That is, the control unit 121 causes the inference parameter to be learned using only the triaxial angular velocities and the triaxial accelerations supplied from the sensor unit 102 with a small error and a predetermined accuracy or more within the range surrounded by the solid line in FIG. 2.

As a result, the posture, speed, and position of the sensor unit 102 can be inferred with high accuracy by the inference parameters obtained by learning and the input of the triaxial angular velocities and the triaxial accelerations supplied from the sensor unit 102.

Note that, as will be described later, in learning, when the triaxial angular velocities and the triaxial accelerations supplied from the sensor unit 102 that do not satisfy the predetermined accuracy are supplied, the output unit 123 may be caused to present the fact to inquire whether or not to use the input for learning, and when the input unit 124 including a keyboard, an operation button, or the like is operated and an instruction not to use the input for learning is not given, the learning may be continued, and when an instruction not to use the input for learning is given, the learning may be stopped.

The control unit 121 includes a posture calculation unit 151, a speed calculation unit 152, a position calculation unit 153, a label input possibility determination unit 154, a learning device 155, a learning recording unit 156, and an inference device 157.

The posture calculation unit 151 has a configuration corresponding to the posture calculation unit 51 in FIG. 1, and when acquiring the triaxial angular velocities in the sensor coordinate system supplied from the sensor unit 102 and the time information (time stamp) of the detection result, integrates the triaxial angular velocities to obtain an angle indicating the posture in the global coordinate system indicating the posture of the sensor unit 102, and outputs the angle to the speed calculation unit 152 and the label input possibility determination unit 154.

When it is determined that there is no movement, the posture calculation unit 151 resets the posture on the basis of the triaxial accelerations. That is, when it is determined that there is no movement, only the gravitational acceleration is substantially detected as the triaxial accelerations, and thus, the posture calculation unit 151 resets the posture on the basis of the gravitational acceleration.

The speed calculation unit 152 has a configuration corresponding to the speed calculation unit 53 in FIG. 1, and converts the triaxial accelerations in the sensor coordinate system into the information in the global coordinate system when acquiring the triaxial accelerations in the sensor coordinate system supplied from the sensor unit 102, the time information of the corresponding detection result, and the information of the angle indicating the posture in the global coordinate system supplied from the posture calculation unit 151. Then, the speed calculation unit 152 obtains the speed in the global coordinate system of the sensor unit 102 by integrating the triaxial accelerations in the global coordinate system of the sensor unit 102, and outputs the speed to the position calculation unit 153 and the label input possibility determination unit 154.

When acquiring the speed information in the global coordinate system supplied from the speed calculation unit 152 and the corresponding time information, the position calculation unit 153 obtains (coordinates of) the position in the global coordinate system of the sensor unit 102 by integrating the speed in the global coordinate system, and outputs the position to the label input possibility determination unit 154.

When acquiring the information of the angle indicating the posture supplied from the posture calculation unit 151, the information of the speed supplied from the speed calculation unit 152, the information of the position supplied from the position calculation unit 153, and the corresponding time information, the triaxial angular velocities and the triaxial accelerations in the sensor coordinate system supplied from the sensor unit 102, the label input possibility determination unit 154 determines whether or not the posture, the speed, and the position are information that can be input as a learning label on the basis of whether or not the posture, the speed, and the position are information with higher accuracy than predetermined accuracy that can be used for learning of the inference parameter.

The information of the posture, speed, and position with higher accuracy than the predetermined accuracy that can be used for learning is, for example, information with relatively small accumulation of errors within the range surrounded by the solid line in FIG. 2.

More specifically, the information of the posture, the speed, and the position with higher accuracy than the predetermined accuracy that can be used for learning is, for example, information of the posture, the speed, and the position in which the elapsed time since the posture determination flag, which is set to ON when the posture is reset, is set to ON, has not exceeded the predetermined time and relatively errors are not cumulatively accumulated.

Note that the posture determination flag is a flag that is turned ON when the posture is reset. For this reason, in the information of the posture, the speed, and the position obtained on the basis of the triaxial angular velocities and the triaxial accelerations supplied from the sensor unit 102, as the elapsed time after the posture determination flag is turned ON becomes longer, errors are cumulatively accumulated, and the accuracy decreases.

The posture determination flag is set to OFF when the accuracy of the posture, speed, and position information obtained on the basis of the triaxial angular velocities and the triaxial accelerations supplied from the sensor unit 102 decreases, the predetermined accuracy is not satisfied, and the posture determination flag is determined to be inappropriate to be used for learning.

The label input possibility determination unit 154 controls the learning device 155 to set a predetermined number (for example, N in time series) of data and time information that are determined to be the posture, the speed, and the position with higher accuracy than predetermined accuracy that can be used for learning as labels that are correct in learning, execute machine learning with the triaxial angular velocities and the triaxial accelerations in the sensor coordinate system supplied from the corresponding sensor unit 102 as inputs, obtain inference parameters for inferring the posture, the speed, and the position, and record the inference parameters in the learning recording unit 156.

The inference device 157 reads the learned inference parameter stored in the learning recording unit 156, and infers the posture, speed, and position of the sensor unit 102 on the basis of the triaxial angular velocities and the triaxial accelerations in the sensor coordinate system supplied from the sensor unit 102, and outputs the inference result to the output unit 123 to present to the user.

With such a configuration, the learning device 155 can store the inference parameter in the learning recording unit 156 by repeating machine learning in which the posture, the speed, and the position with high accuracy are used as labels and the triaxial angular velocities and the triaxial accelerations in the sensor coordinate system supplied from the corresponding sensor unit 102 are used as inputs.

Note that, in the motion capture system 100 of the present disclosure, after inferring the posture, speed, and position of each of the sensor units 102-1 to 102-6, the inference device 157 further obtains information regarding the relative speed and position of each of the sensor units 102-2 to 102-6 with the sensor unit 102-1 fixed to the body portion as a reference position, as motion of the user H, and outputs and presents the motion to the output unit 123.

As a result, in learning of the inference parameter, personalization corresponding to the habit of the motion of the user H or the like is performed, so that the posture, the speed, and the position can be inferred with higher accuracy.

Furthermore, the sensor units 102-1 to 102-6 are fixed to the body portion, the head, the right and left wrists, and the right and left ankles of the user H, and are capable of detecting minute movements, and thus, for example, it is possible to perform learning with higher accuracy than learning of inference parameters using an image captured by a camera as a correct answer label, and as a result, it is possible to implement motion capture with high accuracy corresponding to minute movements.

### Learning Processing by Electronic Device in FIG. 5

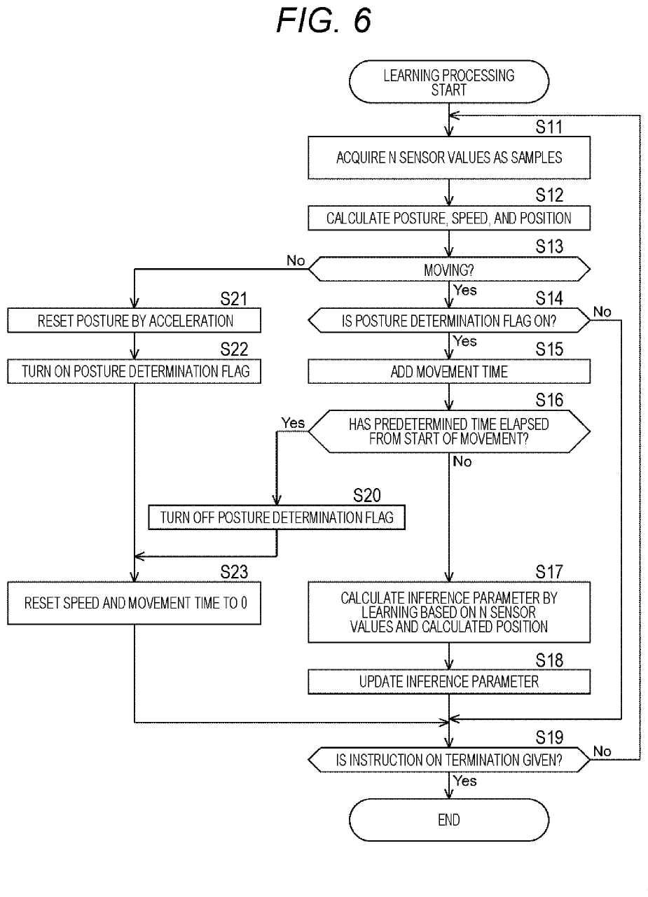

Next, learning processing by the electronic device 101 of FIG. 5 will be described with reference to a flowchart of FIG. 6.

Note that, when the learning processing is performed in the electronic device 101, it is assumed that the triaxial angular velocities in the sensor coordinate system detected by the gyro sensor 172 and the triaxial accelerations in the sensor system coordinate system detected by the acceleration sensor 173 are continuously supplied from the sensor unit 102 via the communication unit 175 in association with the time information (time stamp) output from the RTC 174 at each detection timing at a predetermined time interval.

In other words, it is assumed that the electronic device 101 continues to acquire the information of the triaxial angular velocities and the triaxial accelerations in the sensor coordinate system sequentially attached with the time information (time stamp) from each of the sensor units 102-1 to 102-6 via the communication unit 122 at predetermined time intervals.

Furthermore, hereinafter, the information of the triaxial angular velocities and the triaxial accelerations in the sensor coordinate system supplied from the sensor unit 102 is also simply referred to as a sensor value.

Furthermore, as a default, in the first processing, it is assumed that the posture determination flag is turned ON, and the elapsed time from the start of movement is 0.

Furthermore, learning of the inference parameter of one sensor unit 102 will be described here, but in practice, the electronic device 101 simultaneously executes learning processing of the inference parameter of each of the sensor units 102-1 to 102-6 in parallel. However, since the learning processing of each of the sensor units 102-1 to 102-6 is basically similar, only the processing for one sensor unit 102 will be described.

In step S11, the control unit 121 controls the communication unit 122 to acquire a predetermined number of sensor values supplied from the sensor unit 102 as samples, and supplies information necessary for each of the acquired sensor values to the posture calculation unit 151, the speed calculation unit 152, the position calculation unit 153, the label input possibility determination unit 154, the learning device 155, and the inference device 157.

That is, the communication unit 122 supplies the triaxial angular velocities and accelerations among the sensor values to the learning device 155, the triaxial angular velocities and accelerations and the corresponding time information (time stamps) to the posture calculation unit 151 and the label input possibility determination unit 154, the triaxial accelerations and the corresponding time information (time stamps) to the speed calculation unit 152, and the time information (time stamps) to the position calculation unit 153.

In step S12, the posture calculation unit 151, the speed calculation unit 152, and the position calculation unit 153 calculate the posture, the speed, and the position of the sensor unit 102 corresponding to the sensor value.

More specifically, the posture calculation unit 151 integrates the triaxial angular velocities in the sensor coordinate system, calculates angles indicating the postures of the three axes in the global coordinate system of the sensor unit 102, and outputs the calculated angles to the speed calculation unit 152 and the label input possibility determination unit 154 in association with time information (time stamp).

The speed calculation unit 152 converts the triaxial accelerations in the sensor system coordinate system into the triaxial accelerations in the global coordinate system on the basis of the information of the posture in the global coordinate system supplied from the posture calculation unit 151, and integrates the accelerations to calculate the speed in the global coordinate system, and outputs the speed to the position calculation unit 153 and the label input possibility determination unit 154 in association with the time information (time stamp).

The position calculation unit 153 calculates the position in the global coordinate system by integrating the information of the speed in the global coordinate system, and outputs the position to the label input possibility determination unit 154 in association with the time information (time stamp).

In step S13, the label input possibility determination unit 154 determines whether or not the sensor unit 102 is moving by comparing the information of the position supplied from the position calculation unit 153 with the information of the position supplied immediately before. Note that the presence or absence of movement may be determined on the basis of speed information calculated by the speed calculation unit 152.

In a case where it is determined in step S13 that the user is moving, the processing proceeds to step S14.

In step S14, the label input possibility determination unit 154 determines whether or not the posture determination flag is ON. Here, since this is the first processing, it is determined to be ON, and the processing proceeds to step S15. Note that, in a case where the posture determination flag is OFF in step S14, the processing proceeds to step S19.

In step S15, the label input possibility determination unit 154 adds the elapsed time from the timing at which the sensor value was acquired immediately before as the movement time.

In step S16, the label input possibility determination unit 154 determines whether or not a predetermined time has elapsed from the start of movement. Here, the predetermined time is an elapsed time from the start of movement in which it is considered that an error is accumulated in position information obtained by repeated integration from the start of movement and predetermined accuracy cannot be satisfied, and is, for example, about several seconds.

In a case where it is determined in step S16 that the predetermined time has not elapsed from the start of the movement, that is, in a case where the elapsed time from the start of the movement exceeds the predetermined time, and the information of the position obtained by the repeated integration is regarded as information appropriate as a learning label that satisfies the predetermined accuracy, the processing proceeds to step S17.

In step S17, the label input possibility determination unit 154 regards the information of the position calculated by the position calculation unit 153 as information suitable for the learning label and outputs the information to the learning device 155.

In response to this, the learning device 155 executes machine learning in which the information of the position of the sensor unit 102 supplied from the label input possibility determination unit 154 is used as a learning label, and the triaxial accelerations and the triaxial angular velocities in the sensor coordinate system, which are the sensor values of the sensor unit 102, are used as inputs, and calculates the inference parameter.

In step S18, the learning device 155 updates the inference parameter recorded in the learning recording unit 156 with the inference parameter calculated by machine learning.

In step S19, the control unit 121 determines whether or not an instruction on the end of the processing has been given by operation of the input unit 124 or the like. In a case where it is determined in step S19 that the instruction on the end of the processing is not given, the processing returns to step S11.

Then, in a case where the instruction on the end of the processing is given in step S19, the processing ends.

In addition, in a case where it is determined in step S13 that the moving body is not moving, the processing proceeds to step S21.

In step S21, the posture calculation unit 151 resets the posture on the basis of the triaxial accelerations which are the sensor values.

However, in the case of not moving, the acceleration is only the gravitational acceleration, and thus the roll and the pitch of the angle indicating the posture are reset, but the yaw is not reset.

In step S22, the label input possibility determination unit 154 sets the posture determination flag to ON.

In step S23, the speed calculation unit 152 resets the speed to 0. In addition, at this time, the label input possibility determination unit 154 resets the movement time to 0, and the processing proceeds to step S19.

Furthermore, in step S16, in a case where it is determined that the predetermined time has elapsed from the start of movement, that is, in a case where it is determined that there is a possibility that an error of a predetermined value or more is included in the position information obtained by integration after the predetermined time has elapsed from the start of movement, the processing proceeds to step S20.

In step S20, the label input possibility determination unit 154 turns OFF the posture determination flag so that the machine learning based on the position information calculated by the position calculation unit 153 is not performed, and the processing proceeds to step S23.

Through the series of processing described above, the inference parameter is obtained by machine learning in which the sensor value of the sensor unit 102 is sequentially input along with the movement and the position information obtained on the basis of the sensor value is used as a label, and the processing updated by the inference parameter obtained by the machine learning is repeated.

Then, when it is assumed that the position information obtained by the repeated integration does not satisfy the predetermined accuracy on the basis of the elapsed time from the start of the movement, the posture determination flag is turned OFF and the learning is stopped.

Further, when the movement is stopped, the posture, the speed, and the movement time are reset, and when the movement is started again, the machine learning for calculating the above-described inference parameter is repeated.

As a result, since the machine learning of the inference parameter using the highly accurate position information and the sensor value is repeated, it is possible to obtain the highly accurate inference parameter, and as a result, it is possible to implement the highly accurate inference of the position of the sensor unit 102.

Furthermore, in the above description, by performing machine learning using the sensor value of the sensor unit 102 worn by the user H, the inference parameter corresponding to the motion of the user H is obtained by learning, and personalization of the inference parameter can be implemented. As a result, it is possible to obtain the inference parameter reflecting a habit or the like unique to the user H wearing the sensor unit 102.

As a result, since the inference parameter specialized for the motion of the user H wearing the sensor unit 102 when the inference parameter is learned, in other words, the personalized inference parameter can be obtained by learning, the position when the user H wears the sensor unit 102 can be detected with higher accuracy.

Note that, in the above, an example has been described in which the position information obtained by the position calculation unit 153 is used as a label, and the inference parameter for obtaining the position information of the sensor unit 102 is learned by machine learning using the sensor value as an input.

However, by a similar method, by obtaining the inference parameter with the sensor value as an input by machine learning with at least one of the position information, the speed information, or the posture information of the sensor unit 102 as a label, it is possible to learn the inference parameter that is inferred by personalizing at least one of the position information, the speed information, or the posture information with the sensor value as an input with high accuracy.

Furthermore, in the above description, an example has been described in which the information of the position in the global coordinate system is used as the label used for learning the inference parameter. However, the information of the position in the sensor coordinate system may be used as the label for learning. Note that it similarly applies to the point of using the sensor coordinate system as the label of learning not only for the position but also for the speed.

Furthermore, the processing of learning the inference parameter for inferring the posture, speed, and position of the sensor unit 102 on the basis of the learning label and the sensor value has been described as an example executed in the learning device 155 of the control unit 121 in the electronic device 101. However, the processing may be executed by other configurations, for example, may be executed by a server on a network or cloud computing.

### First Modification of Learning Processing

In the above description, an example has been described in which the learning using the position information as the label by the learning device 155 is stopped and the posture determination flag is turned OFF since the error of the position obtained by the position calculation unit 153 increases due to the cumulative error in a case where the predetermined time has elapsed from the start of the movement. However, after the warning about the decrease in the accuracy of the position is issued, it may be inquired whether or not to use the position for learning, and the learning may be continued until an instruction not to use the position for learning is given.

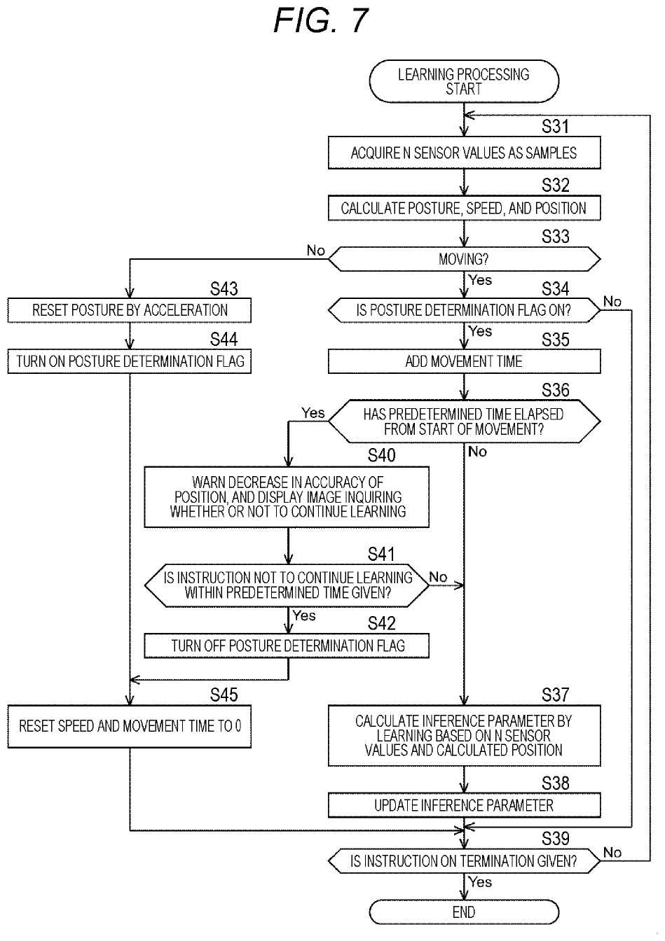

Here, with reference to the flowchart of FIG. 7, a description will be given of learning processing in which, in a case where a predetermined time has elapsed from the start of movement, an inquiry is made as to whether or not to continue learning with the position as a label after warning of a decrease in accuracy of the position, and the learning is continued until an instruction not to continue the learning is given.

Note that steps S31 to S39 and S42 to S45 in the flowchart of FIG. 7 are similar to steps S11 to S23 in the flowchart of FIG. 6, and thus description thereof is omitted.

That is, by the processing of steps S31 to S36, when it is determined that the user is moving, it is determined that the posture determination flag is ON, it is further considered that a predetermined time has elapsed from the start of the movement, and the movement time is added, the processing proceeds to step S40.

In step S40, the label input possibility determination unit 154 controls the output unit 123 to warn that a predetermined time has elapsed from the start of movement, and there is a possibility that the accuracy of the position obtained by the position calculation unit 153 may decrease, and displays an image inquiring whether or not to continue learning using the position as a label.

In step S41, the label input possibility determination unit 154 determines whether or not the input unit 124 is operated within a predetermined time after displaying the images of the warning and the inquiry, and the instruction not to continue the learning with the position as the label is given.

In step S41, in a case where the input unit 124 is operated within a predetermined time after the warning and inquiry images are displayed and the instruction not to continue the learning with the position as the label is not given, it is regarded that continuation of the learning with the position as the label is accepted, and the processing proceeds to step S37. That is, in this case, learning of the inference parameter is continued.

On the other hand, in a case where the input unit 124 is operated within a predetermined time after the warning and inquiry images are displayed and the instruction not to continue the learning with the position as the label is given in step S41, it is regarded that the instruction not to continue the learning with the position as the label is given, and the processing proceeds to step S42.

That is, in this case, the learning of the inference parameter is stopped, and the posture determination flag is set to OFF.

By this processing, even when a predetermined time has elapsed from the start of movement, a warning is given about a decrease in the accuracy of the position, but the learning is continued until the instruction not to continue the learning is given.

As a result, for a motion or the like in which the change in position is relatively small and the error generated by repeating the integration is expected to be small, the learning time can be lengthened by the determination of the user, so that the number of times of learning of the inference parameter can be improved even in a relatively short time.

### Second Modification of Learning Processing

In the above description, an example has been described in which, even in a case where a predetermined time has elapsed from the start of movement, after warning about a decrease in accuracy of a position, an inquiry is made as to whether or not to use the position for learning, and learning is continued until the instruction not to use the position for learning. However, the learning may be stopped in a case where the accuracy of the sensor value used as the input in the machine learning decreases regardless of the elapsed time from the start of the movement.

For example, it is known that regarding the information of the angle indicating the posture of the sensor unit 102, when a posture change of a predetermined angle or more occurs, accuracy decreases regardless of the elapsed time from the start of movement. More specifically, it is known that the accuracy decreases when the change in posture exceeds 360 degrees.

Therefore, the learning may be stopped depending on whether or not the posture change exceeds 360 degrees.

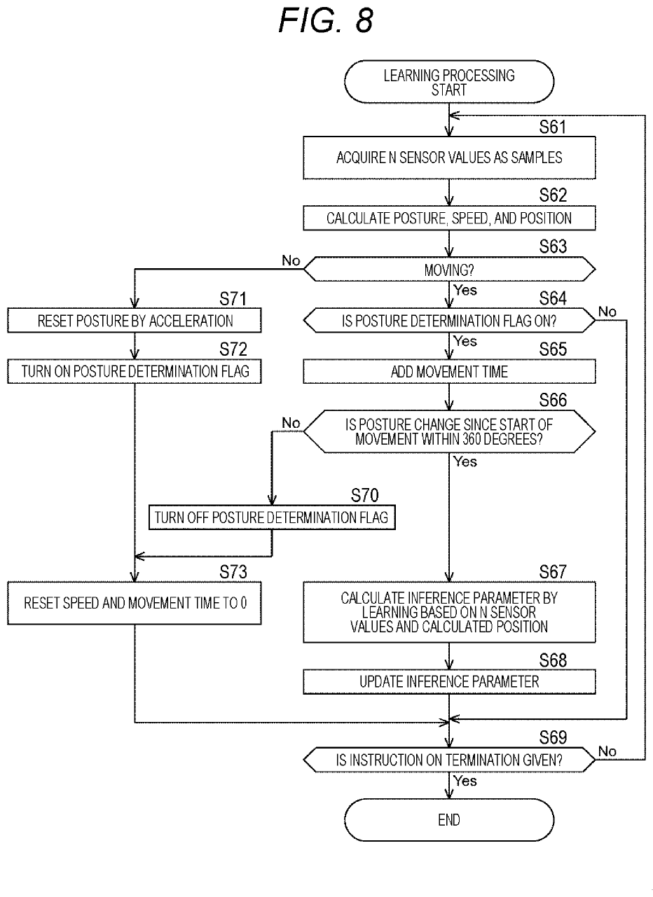

Here, learning processing in which the learning is not continued in a case where the posture change becomes 360 degrees or more will be described with reference to the flowchart of FIG. 8.

Note that the processing of steps S61 to S65 and S67 to S73 in the flowchart of FIG. 8 is similar to the processing of steps S11 to S15 and steps S17 to S23 in the flowchart of FIG. 6, and thus the description thereof will be omitted.

That is, when it is determined by the processing of steps S61 to S65 that the user is moving, it is determined that the posture determination flag is ON, and the movement time is further added, the processing proceeds to step S66.

In step S66, the label input possibility determination unit 154 determines whether or not the posture change calculated by the posture calculation unit 151 is within 360 degrees.

In a case where it is determined in step S66 that the posture change obtained by the posture calculation unit 151 is within 360 degrees, it is assumed that there is no error in the posture calculated by the posture calculation unit 151, and the processing proceeds to step S67. That is, in this case, learning of the inference parameter is continued.

On the other hand, in a case where it is determined in step S66 that the posture change obtained by the posture calculation unit 151 is not within 360 degrees, it is assumed that a decrease in accuracy has occurred in the posture information calculated by the posture calculation unit 151, and the processing proceeds to step S70.

That is, in this case, the learning of the inference parameter is stopped, and the posture determination flag is set to OFF.

By this processing, in a case where it is determined that the posture change exceeds 360 degrees and the accuracy of the posture information calculated by the posture calculation unit 151 decreases, the learning is stopped. As a result, since the learning of the inference parameter using the posture including the error is stopped, it is possible to learn the highly accurate inference parameter.

Note that the determination on the stop of learning based on the elapsed time from the start of movement described above and the determination on whether or not the posture change is within 360 degrees may be combined.

In this manner, it is possible to learn the inference parameter with higher accuracy by determining the stop of learning according to the accuracy of the information used for the label of learning.

### Inference Processing by Electronic Device in FIG. 5

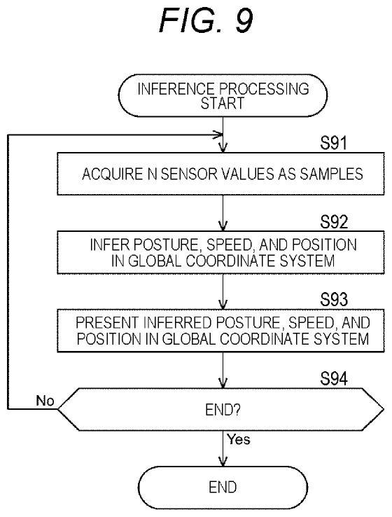

Next, inference processing by the electronic device 101 of FIG. 5 for inferring the posture, speed, and position using the inference parameters obtained by the above-described learning processing will be described with reference to a flowchart of FIG. 9.

In step S91, the inference device 157 acquires, as samples, sensor values including triaxial angular velocities and triaxial accelerations in the sensor coordinate system supplied from a predetermined number (N) of sensor units 102.

In step S92, the inference device 157 reads the inference parameter stored in the learning recording unit 156, and infers the posture, speed, and position in the global coordinate system of the sensor unit 102 on the basis of the triaxial angular velocities and the triaxial accelerations in the sensor coordinate system supplied from the sensor unit 102.

In step S93, the inference device 157 outputs the inferred posture, speed, and position in the global coordinate system of the sensor unit 102 to the output unit 123 to present to the user.

In step S94, the control unit 121 determines whether or not the input unit 124 is operated and an instruction to end the processing is given, and in a case where it is determined that the instruction to end the processing is not given, the processing returns to step S91, and the subsequent processing is repeated.

Then, in step S94, when the input unit 124 is operated and the instruction on termination is given, the processing is terminated.

By the above processing, the posture, speed, and position in the global coordinate system based on the triaxial angular velocities and the triaxial accelerations in the sensor coordinate system supplied from the sensor unit 102 are inferred using the inference parameters learned by the label with high accuracy, so that it is possible to infer the posture, speed, and position in the global coordinate system with high accuracy.

In the motion capture system 100 of FIGS. 3 to 5, after inferring the posture, speed, and position in the global coordinate system of each of the sensor units 102-1 to 102-6, the inference device 157 further obtains the relative speed and position of each of the sensor unit 102-2 fixed to the head, the sensor units 102-3 and 102-4 fixed to the right and left wrists, and the sensor units 102-5 and 102-6 fixed to the right and left ankles with respect to the position of the sensor unit 102-1 fixed to the body portion, and presents the obtained relative speed and position to the output unit 123.

As a result, it is possible to implement highly accurate motion sensing including the relative posture, speed, and position of the head, the right and left wrists, and the right and left ankles with respect to the body portion by reflecting the motion unique to the user H.

Furthermore, since the inference parameter obtained by reflecting the motion of the user H is used, motion sensing including the relative posture, speed, and position of the head, the right and left wrists, and the right and left ankles with respect to the body portion can be implemented in a personalized manner.

As a result, markers are attached to the head, the right and left wrists, the right and left ankles, and the like, and it is possible to detect even fine movements that cannot be implemented by motion sensing or the like based on an image captured by a camera or the like. Therefore, for example, it is possible to detect highly accurate movements including minute twists, vibrations, and the like of hands or feet that are difficult to confirm from the image.

### Modification of Inference Processing

In the above, an example has been described in which the position and speed in the global coordinate system are obtained from the sensor value by using the inference parameter obtained in a case where the position and speed to be the label are in the global coordinate system. However, the position and speed in the global coordinate system may be converted from posture information in the global coordinate system obtained from the triaxial angular velocities after the position and speed in the sensor coordinate system are once obtained from the sensor values by using the inference parameters learned by using the position and speed in the sensor coordinate system as the labels.

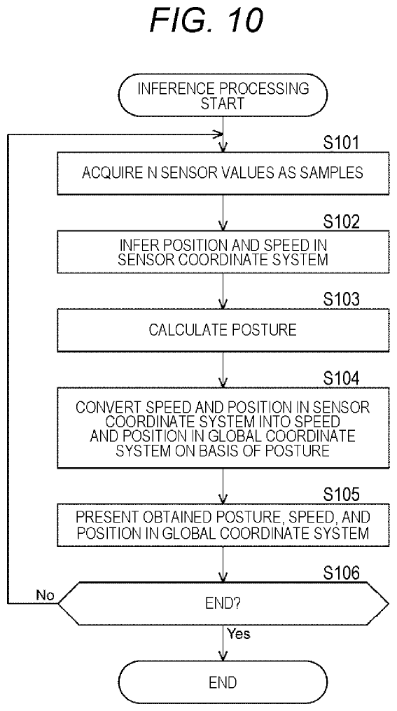

Here, with reference to the flowchart of FIG. 10, a modification of the inference processing will be described in which the position and speed in the sensor coordinate system are once obtained from the sensor values by using the inference parameters learned by using the position and speed in the sensor coordinate system as labels, and then the position and speed in the global coordinate system are converted from posture information in the global coordinate system obtained from the triaxial angular velocities.

In step S101, the inference device 157 acquires, as samples, sensor values including triaxial angular velocities and triaxial accelerations in the sensor coordinate system supplied from a predetermined number (N) of sensor units 102.

In step S102, the inference device 157 reads the inference parameter learned using the label in the sensor coordinate system stored in the learning recording unit 156, and infers the speed and position in the sensor coordinate system of the sensor unit 102 on the basis of the sensor value including the triaxial angular velocities and the triaxial accelerations in the sensor coordinate system supplied from the sensor unit 102.

In step S103, the inference device 157 calculates the posture on the basis of the triaxial angular velocities in the sensor coordinate system supplied from the sensor unit 102. Note that, here, the inference device 157 basically calculates the posture in the global coordinate system of the sensor unit 102 by a method similar to the processing in the posture calculation unit 151.

In step S104, the inference device 157 converts the inferred speed and position in the sensor coordinate system of the sensor unit 102 into the speed and position in the global coordinate system by the posture.

In step S105, the inference device 157 outputs the obtained posture, speed, and position in the global coordinate system of the sensor unit 102 to the output unit 123 to present to the user.

In step S106, the control unit 121 determines whether or not the input unit 124 is operated and an instruction to end the processing is given, and in a case where it is determined that the instruction to end the processing is not given, the processing returns to step S101, and the subsequent processing is repeated.

Then, in step S106, when the input unit 124 is operated and the instruction on termination is given, the processing is terminated.

Also in the above processing, the posture, the speed, and the position in the global coordinate system based on the triaxial angular velocities and the triaxial accelerations in the sensor coordinate system supplied from the sensor unit 102 are inferred using the label in the sensor coordinate system with high accuracy and the inference parameter learned by the input, so that the posture, the speed, and the position in the global coordinate system with high accuracy can be inferred.

### 3. Second Embodiment

In the above description, an example has been described in which, in the electronic device 101, the inference parameter is learned using only the inference parameter with predetermined accuracy among the posture, the speed, and the position obtained on the basis of the sensor value supplied from the sensor unit 102 as a label, thereby implementing highly accurate motion sensing.

However, a label having a predetermined accuracy is often obtained only under a limited condition, such as within a predetermined time from the start of movement or a posture change of 360 degrees or less, and there is a possibility that inference parameters corresponding to various movements actually required cannot be learned.

Therefore, each of the inference parameters may be stored in the learning recording unit 156 for each motion pattern classified into a predetermined number in advance, and the operation corresponding to the motion classified into the unregistered motion pattern may be prompted to the user, and the inference parameters corresponding to various motion patterns may be learned and registered in the learning recording unit 156.

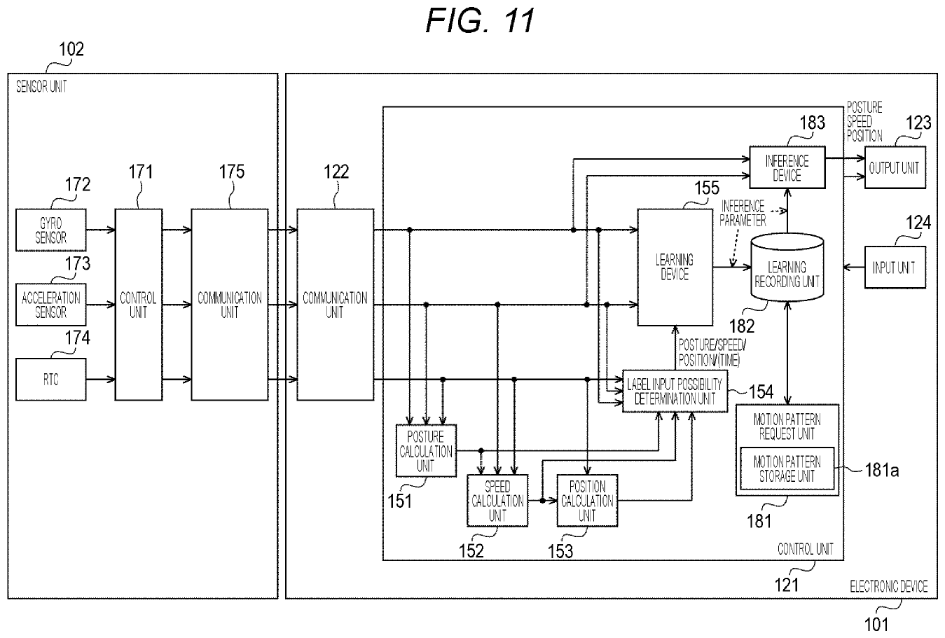

FIG. 11 illustrates a configuration example of the electronic device 101 in which each of the inference parameters is stored in the learning recording unit 156 for each motion pattern classified into a predetermined number in advance, and for a motion pattern for which the inference parameter is not registered, a corresponding operation is prompted to the user, and the inference parameter corresponding to various motion patterns can be learned and registered in the learning recording unit 156.

Note that, in the electronic device 101 in FIG. 11, configurations having functions similar to those of the electronic device 101 in FIG. 5 are denoted by the same reference signs, and the description thereof will be omitted as appropriate.

The electronic device 101 of FIG. 11 is different from the electronic device 101 of FIG. 5 in that a learning recording unit 182 and an inference device 183 are provided instead of the learning recording unit 156 and the inference device 157, and a motion pattern request unit 181 is newly provided.

The learning recording unit 182 has a basic function similar to that of the learning recording unit 156, but the registered inference parameter is registered for each of the plurality of motion patterns registered in advance.

The inference device 183 is basically similar to the function of the inference device 157, but reads the inference parameter from the learning recording unit 182, infers the posture, the speed, and the position on the basis of the sensor value, and presents the posture, the speed, and the position to the output unit 123.

The motion pattern request unit 181 includes a motion pattern storage unit 181a that stores a plurality of preset types of motion patterns, and searches for an inference parameter corresponding to an unregistered motion pattern and an inference parameter for which learning is insufficient among the inference parameters registered in the learning recording unit 182 when learning the inference parameter.

The inference parameter for which learning is insufficient here is, for example, an inference parameter of a motion pattern in which the number of times of learning is lower than a predetermined number of times.

In order to learn the inference parameter of the searched motion pattern, the motion pattern request unit 181 controls the output unit 123 to present information prompting the user to perform a corresponding operation, requests the user to perform the corresponding operation, and then learns the inference parameter of the corresponding motion pattern. In accordance with the motion pattern, the motion pattern request unit 181 presents information for prompting the user to perform a motion of taking a predetermined pose, a motion of raising an arm, a motion of raising a foot, a motion of kicking a ball, a motion of hitting a ball with a racket, or the like, for example.

### Learning Processing by Electronic Device in FIG. 11

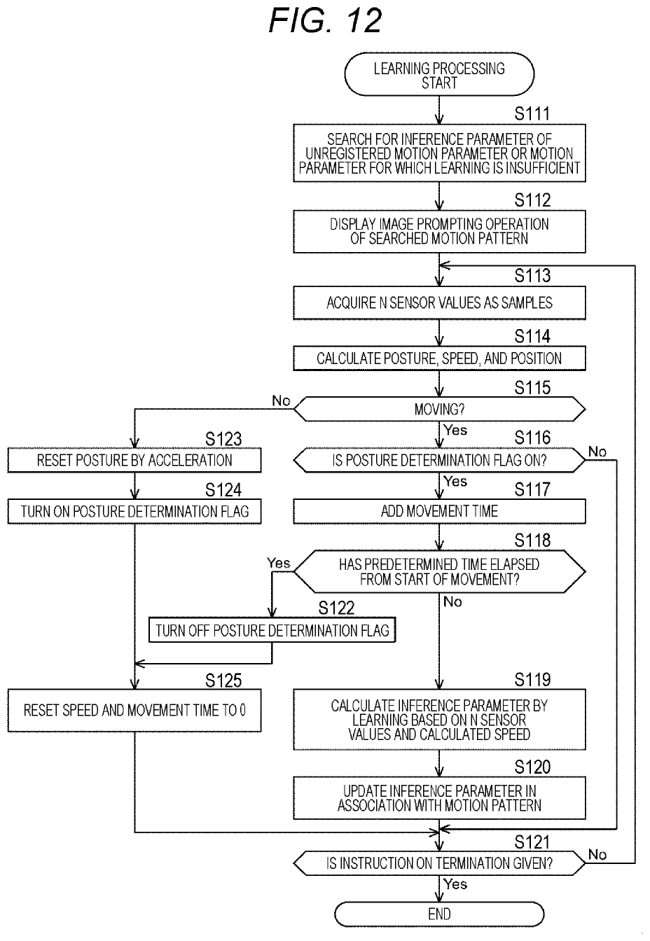

Next, learning processing by the electronic device 101 in FIG. 11 will be described with reference to a flowchart in FIG. 12. Here, an example of learning the inference parameter using the speed calculated by the speed calculation unit 152 as a label instead of the position calculated by the position calculation unit 153 as a label will be described.

Note that the processing of steps S113 to S118 and S121 to S125 in the flowchart of FIG. 12 is similar to the processing of steps S11 to S16 and steps S19 to S23 in the flowchart of FIG. 6, and thus description thereof is omitted.

That is, in step S111, the motion pattern request unit 181 accesses the learning recording unit 182, and searches for an inference parameter of an unregistered motion parameter and a motion parameter for which learning is insufficient among the inference parameters corresponding to the motion patterns registered in advance in the motion pattern storage unit 181a.

In step S112, the motion pattern request unit 181 controls the output unit 123 to display an image prompting the user to perform an operation corresponding to the searched unregistered motion pattern and the inference parameter of the motion pattern for which learning is insufficient, and requests the user to perform the corresponding operation.

By this processing, the user makes a corresponding motion, and the inference parameter of the corresponding motion pattern is learned by the processing of steps S113 to S119.

In step S119, the label input possibility determination unit 154 regards the speed information calculated by the speed calculation unit 152 as information suitable for the learning label and outputs the information to the learning device 155.

In response to this, the learning device 155 executes machine learning in which the speed information of the sensor unit 102 supplied from the label input possibility determination unit 154 is used as a label and the acceleration and the angular velocity in the sensor coordinate system which are the sensor values of the sensor unit 102 are used as inputs, and calculates the inference parameter.

Then, in step S120, the learning device 155 updates the inference parameter recorded in the learning recording unit 182 with the inference parameter calculated by the machine learning. At this time, the motion pattern request unit 181 registers the newly updated inference parameter in association with the information of the corresponding motion pattern.

By the above processing, the inference parameters corresponding to the preset motion patterns are learned and registered in the learning recording unit 182, and thus, it is possible to implement highly accurate motion sensing on the basis of the posture, speed, and position of the sensor unit 102 equipped on the user H according to the motion pattern.

Note that, although the example applied to the motion sensing has been described here, for example, camera shake correction of the imaging apparatus may be implemented according to the motion detected in the motion sensing.

In this case, it is possible to cause the user to learn the inference parameter according to the hand habits of the user when imaging the predetermined subject by prompting the operation of repeatedly imaging the specific subject as the motion pattern.

As a result, as the imaging of the specific subject is repeated, the learning accuracy of the inference parameter is improved, so that the posture, speed, and position of the imaging apparatus can be inferred with high accuracy at the time of imaging of the specific subject, and more accurate camera shake correction can be implemented on the basis of the inference result.

By implementing the highly accurate camera shake correction in this manner, for example, it is possible to correct the camera shake in the long-time exposure imaging at dark night with high accuracy, so that it is possible to implement beautiful imaging in which the influence of the camera shake is reduced even in the long-time exposure imaging.

Furthermore, over-learning of the inference parameter in the corresponding posture may be implemented by repeating imaging in the same posture.

Furthermore, by learning the inference parameter during imaging in a state in which a constraint condition that the user is stationary is applied as the intention of the user, for example, learning of the inference parameter using only a posture change as a correct answer label may be performed.

Furthermore, each inference parameter associated with the barycentric position of the imaging apparatus that changes as the lens is replaced may be learned.

Furthermore, since the inference processing is similar to the processing described above, the description thereof will be omitted.

### 4. Third Embodiment

In the above, an example has been described in which when it is recognized that there is no movement and the user is stopped, learning of the inference parameter is repeated by resetting the posture by acceleration, turning ON the posture determination flag, and resetting the speed and the movement time to zero.

However, when the posture, the speed, and the position used as the label are assumed to be lower than the predetermined accuracy and include an error, and the posture determination flag is turned OFF, the posture is not reset unless the user is in a stopped state in which there is no movement, and the posture determination flag also remains OFF, so that the learning cannot be continued.

Therefore, in a case where a continuous operation such as walking is repeated, the speed may be measured using, for example, a global navigation satellite system (GNSS) within an initial predetermined time, and the measured speed may be used thereafter.

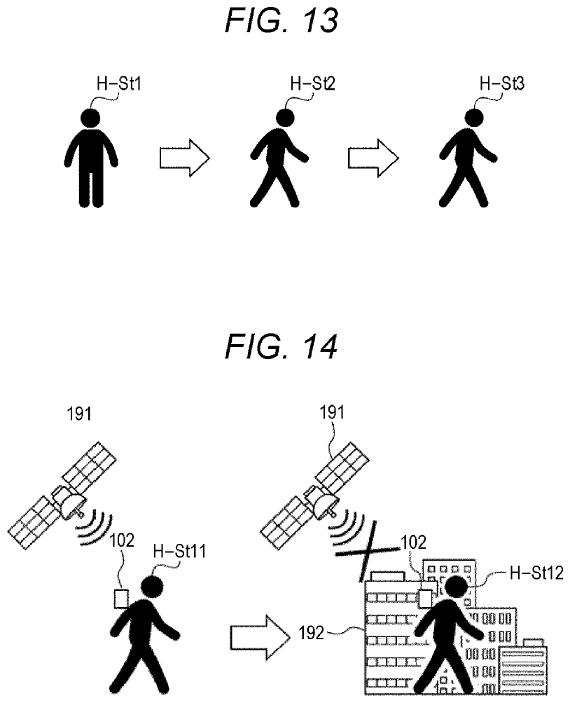

That is, for example, as illustrated in FIG. 13, in a case where the user H-St1 in the first state that is the stationary state starts to move as the user H-St2 in the second state that is the walking state, the speed V1 is obtained using, for example, GNSS or the like immediately after starting to move from the user H-St1 to the user H-St2 in the second state, and the learning of the inference parameter is started.

Thereafter, in a case where the walking as the repeated operation similar to that of the user H-St2 in the second state is continued and the user H-St3 in the third state is reached, for example, the learning of the inference parameter is continued using the speed V1 obtained at the initial stage even when a predetermined time has elapsed since the start of the movement .

As described above, since the posture, the speed, and the position obtained by the posture calculation unit 151, the speed calculation unit 152, and the position calculation unit 153 are obtained by integration, the accuracy decreases as the motion time is continued. Therefore, only information until a predetermined time elapses from the start of the motion or information until the posture change increases to some extent can be used for learning as a label.

For this reason, the posture cannot be reset until the state becomes the stationary state, but in the case of the repeated operation, in particular, since the change in the speed is small, the error is less likely to be accumulated even if the integration is repeated.

Therefore, in a case where the repeated operation is performed in this manner, the learning can be continued by measuring the speed by GNSS or the like for a predetermined time and then using the speed as the measurement result.

As a result, for example, as illustrated in FIG. 14, when the speed is measured on the basis of the information from the GNSS satellite 191, the user H-St11 equipped with the sensor unit 102 capable of measuring the position and the speed by the GNSS continues walking as a repeated operation, and as illustrated by the user H-St12, even in a situation where the sensor unit 102 is surrounded by a high-rise building 192 or the like and cannot receive the information from the GNSS satellite 191 and the user is not in a stop state, it is possible to continue the learning processing based on the speed measured in advance.

### Configuration Example of Sensor Unit and Electronic Device Using GNSS

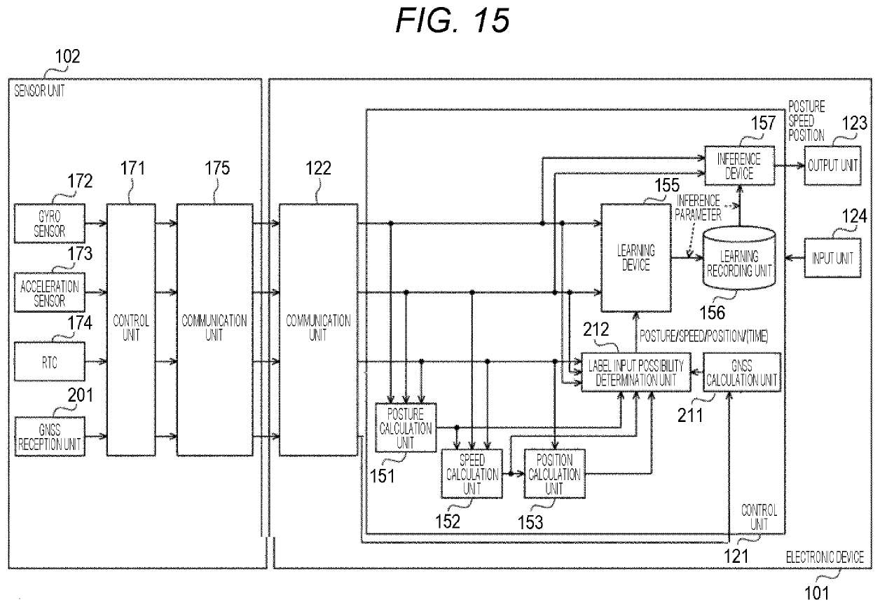

FIG. 15 is a configuration example of the electronic device 101 in which, in a case where the user carrying the sensor unit 102 capable of positioning a position and a speed by the GNSS and the electronic device 101 repeats an operation at a substantially constant speed such as walking, learning of the inference parameter is measured without stopping by using the speed measured using the GNSS.

Note that, in the electronic device 101 and the sensor unit 102 in FIG. 15, configurations having the same functions as those of the electronic device 101 and the sensor unit 102 in FIG. 5 are denoted by the same reference signs, and the description thereof will be appropriately omitted.

That is, the sensor unit 102 in FIG. 15 is different from the sensor unit 102 in FIG. 5 in that a GNSS reception unit 201 is newly provided, and the electronic device 101 is different from the electronic device 101 in FIG. 5 in that a GNSS calculation unit 211 is newly provided, and a label input possibility determination unit 212 is provided instead of the label input possibility determination unit 154.

The GNSS reception unit 201 receives a GNSS signal transmitted from a GNSS satellite (not illustrated), and outputs the GNSS signal to the control unit 171. The control unit 171 outputs the GNSS signal to the electronic device 101 via the communication unit 175 in addition to the triaxial angular velocities, the triaxial accelerations, and the time information (time stamp). The communication unit 122 of the electronic device 101 outputs the newly supplied GNSS signal to the GNSS calculation unit 211.

The GNSS calculation unit 211 is a configuration provided in the control unit 121, calculates the speed and the position on the basis of the reception result of the signal transmitted from the GNSS satellite received by the GNSS reception unit 201, and outputs the speed and the position to the label input possibility determination unit 212.

The label input possibility determination unit 212 basically has a function similar to that of the label input possibility determination unit 154, but further acquires speed and position information supplied from the GNSS calculation unit 211 and uses the information for learning the inference parameter.

Even in a case where the movement is continued, the label input possibility determination unit 212 obtains the absolute posture of the sensor unit 102 on the basis of the speed obtained by the GNSS and the triaxial angular velocities of the sensor values, and resets the posture according to the obtained absolute posture even in a case where an error is cumulatively accumulated after a predetermined time elapses when it is assumed that an operation such as walking is repeatedly performed at a substantially constant speed. As a result, the posture can be reset even in a non-stationary state, and learning can be continued.

### Learning Processing by Electronic Device in FIG. 15

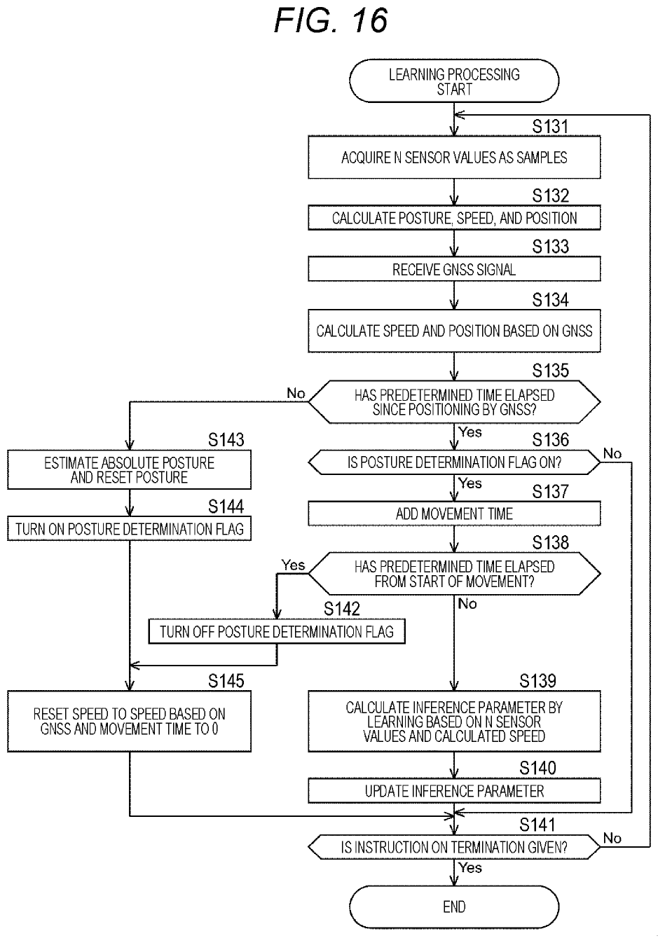

Next, learning processing by the electronic device 101 in FIG. 15 will be described with reference to a flowchart in FIG. 16.

In step S131, the control unit 121 controls the communication unit 122 to acquire a predetermined number (N) of sensor values supplied from the sensor unit 102 as samples, and supplies information necessary for each of the acquired sensor values to the posture calculation unit 151, the speed calculation unit 152, the position calculation unit 153, the label input possibility determination unit 212, the learning device 155, and the inference device 157.

In step S132, the posture calculation unit 151, the speed calculation unit 152, and the position calculation unit 153 calculate the posture, the speed, and the position of the sensor unit 102 corresponding to the sensor value.

In step S133, the GNSS calculation unit 211 acquires a GNSS signal from a GNSS satellite (not illustrated) supplied from the GNSS reception unit 201 of the sensor unit 102.

In step S134, the GNSS calculation unit 211 calculates the position and speed of the sensor unit 102 on the basis of a GNSS signal from a GNSS satellite (not illustrated) received by the GNSS reception unit 201, and outputs the position and speed to the label input possibility determination unit 212.

In step S135, the label input possibility determination unit 212 determines whether or not a predetermined time has elapsed since the position and speed were measured by the GNSS. Here, it is determined whether or not a time until the position and the speed by the GNSS are obtained with predetermined accuracy has elapsed, and for example, it is determined whether or not a time of about several seconds has elapsed.

In a case where it is determined in step S135 that the predetermined time has not elapsed since the position and speed measurement by the GNSS is started, the processing proceeds to step S143.

In step S143, the label input possibility determination unit 212 obtains the absolute posture of the sensor unit 102 on the basis of the speed measured by the GNSS and the triaxial angular velocities of the sensor value, and resets the posture according to the obtained absolute posture.

In step S144, the label input possibility determination unit 212 sets the posture determination flag to ON.

In step S145, the label input possibility determination unit 212 resets the speed calculated by the speed calculation unit 152 at the speed measured by the GNSS. In addition, at this time, the label input possibility determination unit 212 resets the movement time to 0, and the processing proceeds to step S141.

In step S141, the control unit 121 determines whether or not the instruction on the end of the processing has been given by operation of the input unit 124 or the like. In a case where it is determined in step S141 that the instruction on the end of the processing is not given, the processing returns to step S131.

Then, in a case where the instruction on the end of the processing is given in step S141, the processing ends.

That is, when the processing returns to step S131, the speed measured by the GNSS is used as the initial value for the speed calculated by the speed calculation unit 152 thereafter. Then, in a case where it is determined in step S135 that the predetermined time has elapsed since the position and speed measurement by the GNSS is started, the processing proceeds to step S136.

In step S136, the label input possibility determination unit 212 determines whether or not the posture determination flag is ON. Here, since the posture determination flag is turned ON by the above-described processing, the processing proceeds to step S137. Note that, in a case where the posture determination flag is OFF in step S136, the processing proceeds to step S141.

In step S137, the label input possibility determination unit 212 adds the elapsed time from the timing at which the sensor value was acquired immediately before as the movement time.

In step S138, the label input possibility determination unit 212 determines whether or not a predetermined time has elapsed from the start of movement.

In step S138, in a case where it is determined that the predetermined time has not elapsed from the start of the movement, that is, in a case where the elapsed time from the start of the movement exceeds the predetermined time and it is determined that the speed information obtained by the repeated integration does not satisfy the predetermined accuracy and is appropriate information to be used as a label for learning, the processing proceeds to step S139.

In step S139, the label input possibility determination unit 212 regards the speed calculated by the speed calculation unit 152 as information suitable for the learning label and outputs the information to the learning device 155.

In response to this, the learning device 155 executes machine learning in which the speed of the sensor unit 102 supplied from the label input possibility determination unit 212 is used as a label and the acceleration and the angular velocity in the sensor coordinate system, which are sensor values of the sensor unit 102, are used as inputs, and calculates an inference parameter.

In step S140, the learning device 155 updates the inference parameter recorded in the learning recording unit 156 with the inference parameter calculated by machine learning.

In addition, in a case where it is determined in step S138 that the predetermined time has elapsed from the start of the movement, that is, in a case where it is determined that there is a possibility that an error of a predetermined value or more is included in the speed obtained by the integration after the predetermined time has elapsed from the start of the movement, the processing proceeds to step S142.

In step S142, the label input possibility determination unit 212 turns OFF the posture determination flag so that the machine learning based on the speed calculated by the speed calculation unit 152 is not performed, and the processing proceeds to step S145.

According to the above processing, in a case where the user wearing the sensor unit 102 performs an operation repeatedly executed at substantially the same speed such as walking, it is possible to continue learning of the inference parameter without stopping as long as the position and speed are measured by the GNSS.

Note that, although the example in which the speed obtained by the speed calculation unit 152 based on the sensor value is used as a label for learning has been described above, in a case where the speed as a label used for learning may be coarse to some extent (in a case where the number of data per unit time is smaller than a predetermined number), the speed obtained by the GNSS calculation unit 211 may be used as a label for learning.

For example, the walking speed is substantially constant, has a small change, and does not need to capture an abrupt change. Therefore, the number of data per unit time may be less than a predetermined number, and may be coarse. Therefore, the speed obtained by the GNSS calculation unit 211 may be used as a label.

On the other hand, for fine motions of hands and feet, gestures, and the like, it is necessary to detect steep and fine changes, and the number of data per unit time is desirably larger than a predetermined number and dense. Therefore, it is desirable to use a speed obtained by the speed calculation unit 152 on the basis of a sensor value as a label.

### 5. Fourth Embodiment

### Example of Learning Inference Parameters for Predicting Posture, Speed, and Position in Predetermined Time Future

In the above, an example has been described in which the inference parameter for inferring the posture, speed, and position of the sensor unit 102 by the sensor value is learned by learning based on the real-time sensor value of the sensor unit 102 and the posture, speed, and position of the sensor unit 102 obtained by the posture calculation unit 151, the speed calculation unit 152, and the position calculation unit 153.

However, by learning the sensor value of the sensor unit 102 at the timing a predetermined time before and the posture, speed, and position of the sensor unit 102 obtained by the posture calculation unit 151, the speed calculation unit 152, and the position calculation unit 153 on the basis of the real-time sensor value, inference parameters for predicting the future posture, speed, and position by the sensor value for a predetermined time may be learned.

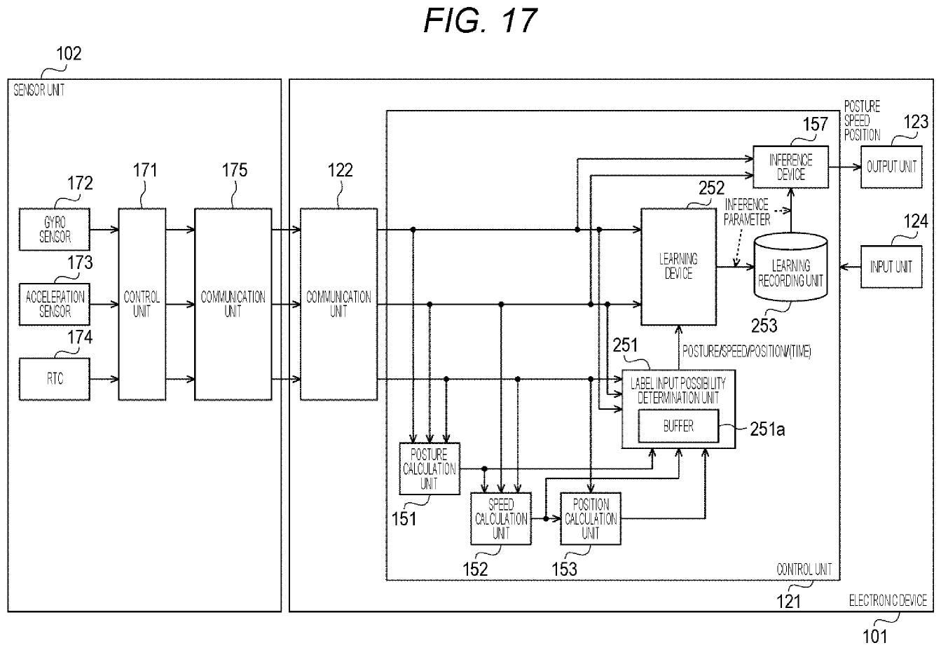

FIG. 17 is a configuration example of the electronic device 101 that learns the inference parameter that predicts the posture, speed, and position in the predetermined time future.

In the electronic device 101 of FIG. 17, configurations having functions similar to those of the electronic device 101 of FIG. 5 are denoted by the same reference signs, and the description thereof will be omitted as appropriate.

That is, the electronic device 101 of FIG. 17 is different from the electronic device 101 of FIG. 5 in that a label input possibility determination unit 251, a learning device 252, and a learning recording unit 253 are provided instead of the label input possibility determination unit 154, the learning device 155, and the learning recording unit 156.

The label input possibility determination unit 251 includes a buffer 251a, buffers a sensor value supplied from the sensor unit 102 in the past for a predetermined time, supplies the posture, the speed, and the position of the sensor unit 102 obtained by the posture calculation unit 151, the speed calculation unit 152, and the position calculation unit 153 and the past sensor value to the learning device 252 on the basis of the real-time sensor value, causes the learning recording unit 253 to learn an inference parameter for inferring the future posture, speed, and position of the sensor unit 102 from the real-time sensor value, and records the inference parameter.

As a result, the inference device 157 can read the inference parameter recorded in the learning recording unit 253 and infer the posture, speed, and position of the sensor unit 102 in the future for a predetermined time using the real-time sensor value.

For example, in a case where mechanical camera shake correction using the posture, speed, and position of the sensor unit 102 is implemented, even if the real-time posture, speed, and position are obtained by inferring them using the inference parameters based on the real-time sensor value, appropriate camera shake correction may not be implemented due to transmission latency by the mechanism.

However, since the future posture, speed, and position are obtained by the sensor value for a predetermined time in this manner, for example, the future posture, speed, and position are inferred and obtained by the time corresponding to the transmission latency, whereby camera shake correction in consideration of the transmission latency can be implemented.

### Learning Processing by Electronic Device in FIG. 17

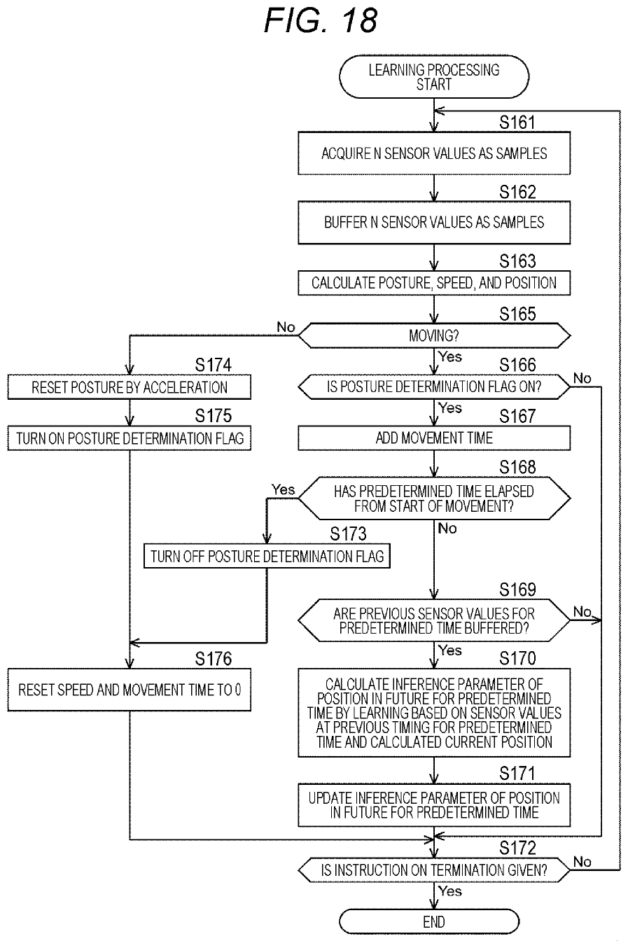

Next, inference parameter learning processing by the electronic device 101 in FIG. 17 will be described with reference to a flowchart in FIG. 18.

Note that, in the flowchart of FIG. 18, the processing of steps S161, S163 to S168 and steps S173 to S176 is similar to the processing of steps S11 to S16 and steps S20 to S23 in the flowchart of FIG. 6, and thus the description thereof will be omitted.

That is, in step S161, when the triaxial angular velocities and accelerations, which are the sensor values supplied from the sensor unit 102, and the corresponding time information (time stamp) are supplied to the label input possibility determination unit 251, the processing proceeds to step S162.

In step S162, the label input possibility determination unit 251 buffers the triaxial angular velocities and accelerations, which are sensor values, and the corresponding time information (time stamp) in the buffer 251a.

Then, by the processing of steps S163 to S168, when the posture, the speed, and the position are calculated, it is determined that the user is moving, the posture determination flag is ON, the movement time is added, and it is determined that the predetermined time has not elapsed from the start of the movement, the processing proceeds to step S169.

In step S169, the label input possibility determination unit 251 determines whether or not the triaxial angular velocities and accelerations, which are the sensor values supplied from the sensor unit 102 for a predetermined time, and the corresponding time information (time stamp) are buffered in the buffer 251a.

In a case where it is determined in step S169 that the triaxial angular velocities and accelerations, which are the sensor values supplied from the sensor unit 102 for the predetermined time, and the corresponding time information (time stamp) are buffered in the buffer 251a, the processing proceeds to step S170. Note that, in a case where it is determined in step S169 that the triaxial angular velocities and accelerations, which are the sensor values supplied from the sensor unit 102 for the predetermined time to the buffer 251a, and the corresponding time information (time stamp) are not buffered, the processing proceeds to step S172.

In step S170, the label input possibility determination unit 251 regards the triaxial angular velocities and accelerations, which are the sensor values supplied from the sensor unit 102, corresponding time information (time stamp), and the position information calculated by the position calculation unit 153 on the basis of the real-time sensor value, buffered in the buffer 251a for a predetermined time, as information suitable for the learning label, and outputs the information to the learning device 252.

In response to this, the learning device 252 executes machine learning in which the real-time position information of the sensor unit 102 calculated by the position calculation unit 153 is used as a label, and the acceleration and the angular velocity in the sensor coordinate system, which are the sensor values of the sensor unit 102 in the past for a predetermined time, supplied from the label input possibility determination unit 251 are used as inputs, and calculates an inference parameter for inferring a future position for a predetermined time.

In step S171, the learning device 252 updates the inference parameter recorded in the learning recording unit 253 with the inference parameter that infers a future position for a predetermined time calculated by machine learning.

By the above processing, since the inference parameter for inferring the future position for a predetermined time is learned, it is possible to infer the future position for a predetermined time with high accuracy from the real-time sensor value.

Although the example in which the position is used as the label used for learning has been described above, it is also possible to obtain an inference parameter for inferring the speed.

Note that the inference processing is similar to the above-described processing using the obtained inference parameter and the real-time sensor value, and thus the description thereof will be omitted.

### 6. Example of Execution by Software

Incidentally, the series of processing described above can be executed by hardware, but can also be executed by software. In a case where the series of processing is executed by software, a program constituting the software is installed from a recording medium into, for example, a computer built into dedicated hardware or a general-purpose computer that is capable of executing various functions by installing various programs, or the like.

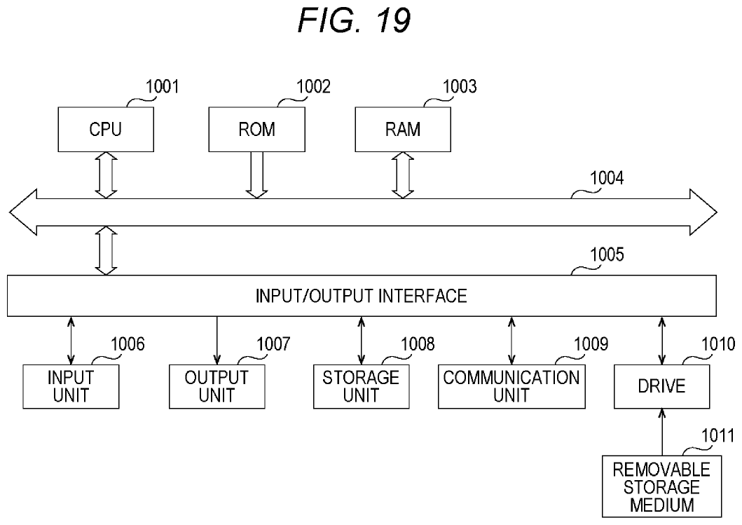

FIG. 19 illustrates a configuration example of a general-purpose computer. This computer includes a central processing unit (CPU) 1001. An input/output interface 1005 is connected to the CPU 1001 via a bus 1004. A read only memory (ROM) 1002 and a random access memory (RAM) 1003 are connected to the bus 1004.

To the input/output interface 1005, an input unit 1006 including an input device such as a keyboard and a mouse by which a user inputs operation commands, an output unit 1007 that outputs a processing operation screen and an image of a processing result to a display device, a storage unit 1008 that includes a hard disk drive and the like and stores programs and various data, and a communication unit 1009 including a local area network (LAN) adapter or the like and executes communication processing via a network represented by the Internet are connected. Furthermore, a drive 1010 that reads and writes data from and to a removable storage medium 1011 such as a magnetic disk (including flexible disk), an optical disk (including compact disc-read only memory (CD-ROM) and digital versatile disc (DVD)), a magneto-optical disk (including Mini Disc (MD)), or a semiconductor memory is connected.

The CPU 1001 executes various processing in accordance with a program stored in the ROM 1002, or a program read from the removable storage medium 1011 such as a magnetic disk, an optical disk, a magneto-optical disk, or a semiconductor memory, installed in the storage unit 1008, and loaded from the storage unit 1008 into the RAM 1003. Furthermore, the RAM 1003 also appropriately stores data necessary for the CPU 1001 to execute various processing, and the like.

In the computer configured as described above, for example, the CPU 1001 loads the program stored in the storage unit 1008 into the RAM 1003 via the input/output interface 1005 and the bus 1004 and executes the program, to thereby perform the above-described series of processing.

The program executed by the computer (CPU 1001) can be provided by being recorded in the removable storage medium 1011 as a package medium or the like, for example. Also, the program can be provided via a wired or wireless transmission medium such as a local area network, the Internet, or digital satellite broadcasting.

In the computer, the program can be installed in the storage unit 1008 via the input/output interface 1005 by mounting the removable storage medium 1011 to the drive 1010. Furthermore, the program can be received by the communication unit 1009 via a wired or wireless transmission medium and installed in the storage unit 1008. In addition, the program can be installed in the ROM 1002 or the storage unit 1008 in advance.

Note that the program to be executed by the computer may be a program that is processed in time series in the order described in the present specification, or may be a program that is processed in parallel or at necessary timings such as when a call is made.

Note that the CPU 1001 in FIG. 19 implements the functions of the control unit 121 in FIGS. 5, 11, 15, and 17.

Furthermore, in the present specification, a system is intended to mean assembly of a plurality of components (apparatuses, modules (parts), and the like) and it does not matter whether or not all the components are in the same housing. Therefore, a plurality of apparatuses accommodated in separate housings and connected via a network and one apparatus in which a plurality of modules is accommodated in one housing are both systems.

Note that embodiments of the present disclosure are not limited to the embodiments described above, and various modifications may be made without departing from the scope of the present disclosure.

For example, the present disclosure can have a configuration of cloud computing in which one function is shared by a plurality of apparatuses via a network and processing is performed in cooperation.

Furthermore, each step described in the above-described flowchart can be executed by one apparatus, and also shared and executed by a plurality of apparatuses.

Moreover, in a case where a plurality of processing is included in one step, the plurality of processing included in the one step can be executed by one apparatus or shared and executed by a plurality of apparatuses.

Note that the present disclosure may have the following configurations.

<1> An information processing apparatus including:

a calculation unit that calculates information to be a learning label on the basis of a sensor value including an angular velocity and an acceleration detected by a sensor unit;

a learning unit that learns an inference parameter for inferring at least one of a posture, a speed, or a position of the sensor unit on the basis of the information to be the learning label calculated by the calculation unit and the sensor value; and

a learning label supply unit that supplies, to the learning unit, information to be the learning label with higher accuracy than predetermined accuracy among the information to be the learning label calculated by the calculation unit,

in which the learning unit learns the inference parameter for inferring at least one of the posture, the speed, or the position of the sensor unit on the basis of the information to be the learning label with higher accuracy than the predetermined accuracy supplied from the learning label supply unit and the sensor value.

<2> The information processing apparatus according to <1>,

in which the calculation unit calculates the information to be the learning label by integrating the sensor value, and

in a case where an elapsed time from start of integration of the sensor value is within a predetermined time, the learning label supply unit supplies the information to be the learning label calculated by the calculation unit to the learning unit as the information to be the learning label with higher accuracy than the predetermined accuracy.

<3> The information processing apparatus according to <2>,

in which the calculation unit includes:

a speed calculation unit that calculates a speed of the sensor unit by integrating the acceleration; and

a position calculation unit that calculates a position of the sensor unit by integrating the speed, and

the learning label supply unit is configured to:

in a case where an elapsed time from start of integration of the acceleration by the speed calculation unit is within a predetermined time, supply information of the speed calculated by the speed calculation unit to the learning unit as the information to be the learning label with higher accuracy than the predetermined accuracy; and

in a case where an elapsed time from start of integration of the speed by the position calculation unit is within a predetermined time, supply information of the position calculated by the position calculation unit to the learning unit as the information to be the learning label with higher accuracy than the predetermined accuracy.

<4> The information processing apparatus according to <3>,

in which, when the sensor unit is stationary, the learning label supply unit resets the information of the speed calculated by the speed calculation unit to 0, and resets the elapsed time from the start of the integration of the acceleration by the speed calculation unit to 0.

<5> The information processing apparatus according to <3>,

in which, in a case where the elapsed time from the start of the integration of the acceleration by the speed calculation unit exceeds a predetermined time or in a case where the elapsed time from the start of the integration of the speed by the position calculation unit exceeds a predetermined time, the learning label supply unit presents that the information of the speed calculated by the speed calculation unit or the information of the position calculated by the position calculation unit is not the information to be the learning label with higher accuracy than the predetermined accuracy.

<6> The information processing apparatus according to <5>,

in which, when presenting, to a user, that the information of the speed calculated by the speed calculation unit or the information of the position calculated by the position calculation unit is not the information to be the learning label with higher accuracy than the predetermined accuracy, the learning label supply unit further presents information for inquiring whether or not to supply the information of the speed calculated by the speed calculation unit or the information of the position calculated by the position calculation unit to the learning unit as the information to be the learning label.

<7> The information processing apparatus according to <6>, further including

an external measurement unit that measures a position and a speed of the sensor unit on the basis of an external signal,

in which, in a case where the external measurement unit measures the position and the speed of the sensor unit for a predetermined time or more, the learning label supply unit resets the information of the speed calculated by the speed calculation unit at the speed measured by the external measurement unit, and resets the elapsed time from the start of the integration of the acceleration by the speed calculation unit to 0.

<8> The information processing apparatus according to <7>,

in which the external measurement unit measures the position and the speed on the basis of a signal from a global navigation satellite system (GNSS) satellite.

<9> The information processing apparatus according to <2>,

in which the calculation unit includes:

a posture calculation unit that calculates an angle to be a posture of the sensor unit by integrating the angular velocity, and

the learning label supply unit is configured to

in a case where a posture change after integration of the angular velocity by the posture calculation unit is started does not exceed a predetermined value, supply information of the posture calculated by the posture calculation unit to the learning unit as the information to be the learning label with higher accuracy than the predetermined accuracy.

<10> The information processing apparatus according to <9>,

in which, when the sensor unit is stationary, the learning label supply unit resets the information of the posture calculated by the posture calculation unit at the acceleration of the sensor value.

<11> The information processing apparatus according to <1>, further including:

a learning recording unit that stores the inference parameter for each of a plurality of preset motion patterns; and

a motion pattern search unit that searches for an inference parameter of an unregistered motion pattern among the inference parameter stored in the learning recording unit,

in which, when causing the learning unit to learn the inference parameter of the unregistered motion pattern, the motion pattern search unit presents information prompting an operation corresponding to the unregistered motion pattern to a user equipped with the sensor unit.

<12> The information processing apparatus according to <11>,

in which the motion pattern search unit presents, as the operation corresponding to the unregistered motion pattern, information prompting the user to perform a motion of taking a predetermined pose, a motion of raising an arm, a motion of raising a foot, a motion of kicking a ball, or a motion of hitting a ball with a racket.

<13> The information processing apparatus according to <1>,

in which the learning label supply unit further includes

a buffering unit that buffers the sensor value for a predetermined time, and

supplies, to the learning unit, information to be a current learning label with higher accuracy than the predetermined accuracy among the information to be the learning label calculated by the calculation unit and the sensor value in a past for a predetermined time buffered in the buffering unit, and

the learning unit learns the inference parameter for inferring at least one of the posture, the speed, or the position of the sensor unit in a future for a predetermined time on the basis of the information to be the current learning label with higher accuracy than the predetermined accuracy supplied from the learning label supply unit and the sensor value in the past for the predetermined time.

<14> The information processing apparatus according to any one of <1> to <13>, further including

an inference device that infers at least one of the posture, the speed, or the position of the sensor unit on the basis of the sensor value including the angular velocity and the acceleration detected by the sensor unit by using the inference parameter learned by the learning unit. <15> The information processing apparatus according to <14>,

in which the calculation unit calculates the information to be the learning label in a global coordinate system on the basis of the sensor value in a sensor coordinate system, and the learning unit learns the inference parameter for

inferring at least one of the posture, the speed, or the position of the sensor unit in the global coordinate system on the basis of the information to be the learning label in the global coordinate system with higher accuracy than the predetermined accuracy supplied from the learning label supply unit and the sensor value in the sensor coordinate system.

<16> The information processing apparatus according to <15>,

in which the inference device infers at least one of the posture, the speed, or the position of the sensor unit in the global coordinate system on the basis of the sensor value of the sensor unit in the sensor coordinate system by using the inference parameter learned by the learning unit.

<17> The information processing apparatus according to <14>,

in which the calculation unit calculates the information to be the learning label in the sensor coordinate system on the basis of the sensor value in the sensor coordinate system, and

the learning unit learns the inference parameter for inferring at least one of the posture, the speed, or the position of the sensor unit in the sensor coordinate system on the basis of the information to be the learning label in the sensor coordinate system with higher accuracy than the predetermined accuracy supplied from the learning label supply unit and the sensor value in the sensor coordinate system.

<18> The information processing apparatus according to <17>,

in which the inference device is configured to:

calculate the posture in the global coordinate system on the basis of the sensor value of the sensor unit in the sensor coordinate system;

infer at least one of the speed or the position of the sensor unit in the sensor coordinate system on the basis of the sensor value of the sensor unit in the sensor coordinate system by using the inference parameter learned by the learning unit; and

convert at least one of the speed or the position of the sensor unit in the sensor coordinate system inferred into at least one of the speed or the position of the sensor unit in the global coordinate system on the basis of the posture in the global coordinate system.

<19> An information processing method of an information processing apparatus including:

a calculation unit;

a learning unit; and

a learning label supply unit,

in which the calculation unit calculates information to be a learning label on the basis of a sensor value including an angular velocity and an acceleration detected by a sensor unit,

the learning unit learns an inference parameter for inferring at least one of a posture, a speed, or a position of the sensor unit on the basis of the information to be the learning label calculated by the calculation unit and the sensor value,

the learning label supply unit supplies, to the learning unit, information to be the learning label with higher accuracy than predetermined accuracy among the information to be the learning label calculated by the calculation unit, and

the learning unit learns the inference parameter for inferring at least one of the posture, the speed, or the position of the sensor unit on the basis of the information to be the learning label with higher accuracy than the predetermined accuracy supplied from the learning label supply unit and the sensor value.

<20> A program for causing a computer to function as:

a calculation unit that calculates information to be a learning label on the basis of a sensor value including an angular velocity and an acceleration detected by a sensor unit;

a learning unit that learns an inference parameter for inferring at least one of a posture, a speed, or a position of the sensor unit on the basis of the information to be the learning label calculated by the calculation unit and the sensor value; and

a learning label supply unit that supplies, to the learning unit, information to be the learning label with higher accuracy than predetermined accuracy among the information to be the learning label calculated by the calculation unit,

in which the learning unit learns the inference parameter for inferring at least one of the posture, the speed, or the position of the sensor unit on the basis of the information to be the learning label with higher accuracy than the predetermined accuracy supplied from the learning label supply unit and the sensor value.

### REFERENCE SIGNS LIST

100 Motion capture system
101 Electronic device
102, 102-1 to 102-6 Sensor unit
121 Control unit
122 Communication unit
123 Output unit
124 Input unit
151 Posture calculation unit
152 Speed calculation unit
153 Position calculation unit
154 Label input possibility determination unit
155 Learning device
156 Learning recording unit
157 Inference device
181 Motion pattern request unit
181a Motion pattern storage unit
201 GNSS reception unit
211 GNSS calculation unit
212 Label input possibility determination unit
251 Label input possibility determination unit
251a Buffer
252 Learning device
253 Learning recording unit

1. An information processing apparatus comprising:
a calculation unit that calculates information to be a learning label on a basis of a sensor value including an angular velocity and an acceleration detected by a sensor unit;
a learning unit that learns an inference parameter for inferring at least one of a posture, a speed, or a position of the sensor unit on a basis of the information to be the learning label calculated by the calculation unit and the sensor value; and
a learning label supply unit that supplies, to the learning unit, information to be the learning label with higher accuracy than predetermined accuracy among the information to be the learning label calculated by the calculation unit,
wherein the learning unit learns the inference parameter for inferring at least one of the posture, the speed, or the position of the sensor unit on a basis of the information to be the learning label with higher accuracy than the predetermined accuracy supplied from the learning label supply unit and the sensor value.

2. The information processing apparatus according to claim 1,
wherein the calculation unit calculates the information to be the learning label by integrating the sensor value, and
in a case where an elapsed time from start of integration of the sensor value is within a predetermined time, the learning label supply unit supplies the information to be the learning label calculated by the calculation unit to the learning unit as the information to be the learning label with higher accuracy than the predetermined accuracy.

3. The information processing apparatus according to claim 2,
wherein the calculation unit includes:
a speed calculation unit that calculates a speed of the sensor unit by integrating the acceleration; and
a position calculation unit that calculates a position of the sensor unit by integrating the speed, and
the learning label supply unit is configured to:
in a case where an elapsed time from start of integration of the acceleration by the speed calculation unit is within a predetermined time, supply information of the speed calculated by the speed calculation unit to the learning unit as the information to be the learning label with higher accuracy than the predetermined accuracy; and
in a case where an elapsed time from start of integration of the speed by the position calculation unit is within a predetermined time, supply information of the position calculated by the position calculation unit to the learning unit as the information to be the learning label with higher accuracy than the predetermined accuracy.

4. The information processing apparatus according to claim 3,
wherein, when the sensor unit is stationary, the learning label supply unit resets the information of the speed calculated by the speed calculation unit to 0, and resets the elapsed time from the start of the integration of the acceleration by the speed calculation unit to 0.

5. The information processing apparatus according to claim 3,
wherein, in a case where the elapsed time from the start of the integration of the acceleration by the speed calculation unit exceeds a predetermined time or in a case where the elapsed time from the start of the integration of the speed by the position calculation unit exceeds a predetermined time, the learning label supply unit presents that the information of the speed calculated by the speed calculation unit or the information of the position calculated by the position calculation unit is not the information to be the learning label with higher accuracy than the predetermined accuracy.

6. The information processing apparatus according to claim 5,
wherein, when presenting, to a user, that the information of the speed calculated by the speed calculation unit or the information of the position calculated by the position calculation unit is not the information to be the learning label with higher accuracy than the predetermined accuracy, the learning label supply unit further presents information for inquiring whether or not to supply the information of the speed calculated by the speed calculation unit or the information of the position calculated by the position calculation unit to the learning unit as the information to be the learning label.

7. The information processing apparatus according to claim 6, further comprising
an external measurement unit that measures a position and a speed of the sensor unit on a basis of an external signal,
wherein, in a case where the external measurement unit measures the position and the speed of the sensor unit for a predetermined time or more, the learning label supply unit resets the information of the speed calculated by the speed calculation unit at the speed measured by the external measurement unit, and resets the elapsed time from the start of the integration of the acceleration by the speed calculation unit to 0.

8. The information processing apparatus according to claim 7,
wherein the external measurement unit measures the position and the speed on a basis of a signal from a global navigation satellite system (GNSS) satellite.

9. The information processing apparatus according to claim 2,
wherein the calculation unit includes:
a posture calculation unit that calculates an angle to be a posture of the sensor unit by integrating the angular velocity, and
the learning label supply unit is configured to
in a case where a posture change after integration of the angular velocity by the posture calculation unit is started does not exceed a predetermined value, supply information of the posture calculated by the posture calculation unit to the learning unit as the information to be the learning label with higher accuracy than the predetermined accuracy.

10. The information processing apparatus according to claim 9,
wherein, when the sensor unit is stationary, the learning label supply unit resets the information of the posture calculated by the posture calculation unit at the acceleration of the sensor value.

11. The information processing apparatus according to claim 1, further comprising:
a learning recording unit that stores the inference parameter for each of a plurality of preset motion patterns; and
a motion pattern search unit that searches for an inference parameter of an unregistered motion pattern among the inference parameter stored in the learning recording unit,
wherein, when causing the learning unit to learn the inference parameter of the unregistered motion pattern, the motion pattern search unit presents information prompting an operation corresponding to the unregistered motion pattern to a user equipped with the sensor unit.

12. The information processing apparatus according to claim 11,
wherein the motion pattern search unit presents, as the operation corresponding to the unregistered motion pattern, information prompting the user to perform a motion of taking a predetermined pose, a motion of raising an arm, a motion of raising a foot, a motion of kicking a ball, or a motion of hitting a ball with a racket.

13. The information processing apparatus according to claim 1,
wherein the learning label supply unit further includes
a buffering unit that buffers the sensor value for a predetermined time, and
supplies, to the learning unit, information to be a current learning label with higher accuracy than the predetermined accuracy among the information to be the learning label calculated by the calculation unit and the sensor value in a past for a predetermined time buffered in the buffering unit, and
the learning unit learns the inference parameter for inferring at least one of the posture, the speed, or the position of the sensor unit in a future for a predetermined time on a basis of the information to be the current learning label with higher accuracy than the predetermined accuracy supplied from the learning label supply unit and the sensor value in the past for the predetermined time.

14. The information processing apparatus according to claim 1, further comprising
an inference device that infers at least one of the posture, the speed, or the position of the sensor unit on a basis of the sensor value including the angular velocity and the acceleration detected by the sensor unit by using the inference parameter learned by the learning unit.

15. The information processing apparatus according to claim 14,
wherein the calculation unit calculates the information to be the learning label in a global coordinate system on a basis of the sensor value in a sensor coordinate system, and
the learning unit learns the inference parameter for inferring at least one of the posture, the speed, or the position of the sensor unit in the global coordinate system on a basis of the information to be the learning label in the global coordinate system with higher accuracy than the predetermined accuracy supplied from the learning label supply unit and the sensor value in the sensor coordinate system.

16. The information processing apparatus according to claim 15,
wherein the inference device infers at least one of the posture, the speed, or the position of the sensor unit in the global coordinate system on a basis of the sensor value of the sensor unit in the sensor coordinate system by using the inference parameter learned by the learning unit.

17. The information processing apparatus according to claim 14,
wherein the calculation unit calculates the information to be the learning label in the sensor coordinate system on a basis of the sensor value in the sensor coordinate system, and
the learning unit learns the inference parameter for inferring at least one of the posture, the speed, or the position of the sensor unit in the sensor coordinate system on a basis of the information to be the learning label in the sensor coordinate system with higher accuracy than the predetermined accuracy supplied from the learning label supply unit and the sensor value in the sensor coordinate system.

18. The information processing apparatus according to claim 17,
wherein the inference device is configured to:
calculate the posture in the global coordinate system on a basis of the sensor value of the sensor unit in the sensor coordinate system;
infer at least one of the speed or the position of the sensor unit in the sensor coordinate system on a basis of the sensor value of the sensor unit in the sensor coordinate system by using the inference parameter learned by the learning unit; and
convert at least one of the speed or the position of the sensor unit in the sensor coordinate system inferred into at least one of the speed or the position of the sensor unit in the global coordinate system on a basis of the posture in the global coordinate system.

19. An information processing method of an information processing apparatus comprising:
a calculation unit;
a learning unit; and
a learning label supply unit,
wherein the calculation unit calculates information to be a learning label on a basis of a sensor value including an angular velocity and an acceleration detected by a sensor unit,
the learning unit learns an inference parameter for inferring at least one of a posture, a speed, or a position of the sensor unit on a basis of the information to be the learning label calculated by the calculation unit and the sensor value,
the learning label supply unit supplies, to the learning unit, information to be the learning label with higher accuracy than predetermined accuracy among the information to be the learning label calculated by the calculation unit, and
the learning unit learns the inference parameter for inferring at least one of the posture, the speed, or the position of the sensor unit on a basis of the information to be the learning label with higher accuracy than the predetermined accuracy supplied from the learning label supply unit and the sensor value.

20. A program for causing a computer to function as:
a calculation unit that calculates information to be a learning label on a basis of a sensor value including an angular velocity and an acceleration detected by a sensor unit;
a learning unit that learns an inference parameter for inferring at least one of a posture, a speed, or a position of the sensor unit on a basis of the information to be the learning label calculated by the calculation unit and the sensor value; and
a learning label supply unit that supplies, to the learning unit, information to be the learning label with higher accuracy than predetermined accuracy among the information to be the learning label calculated by the calculation unit,
wherein the learning unit learns the inference parameter for inferring at least one of the posture, the speed, or the position of the sensor unit on a basis of the information to be the learning label with higher accuracy than the predetermined accuracy supplied from the learning label supply unit and the sensor value.
